(19) United States
(12) Patent Application Publication
(10) Pub. No.: US 2008/0070233 A1
(43) Pub. Date: Mar. 20, 2008

(54) Quantitative analysis and typing of subcellular particles

(75) Inventors: Jan Bieschke, Munich (DE); Armln Goese, Munich (DE); Manfred Eigen, Goettlngen (DE); Hans A. Kretzschmar, Wolfratshausen (DE)

Correspondence Address: JACOBSON HOLMAN PLLC, 400 SEVENTH STREET N.W., SUITE 600, WASHINGTON, DC, 20004, US

(73) Assignee: Evotec OAI AG.

(21) Appl. No.: 11/797,508

(22) Filed: May 3, 2007

Related U.S. Application Data
(63) Continuation of application No. 10/089,233, filed on Feb. 2, 2004, now abandoned.

(30) Foreign Application Priority Data
Sep. 28, 1999 (DE) 199 46 549.0
Mar. 22, 2000 (DE) 100 14 234.6

(51) Int. Cl. C12Q 1/70; C12Q 1/68; G01N 33/53

(52) U.S. Cl. 435/5; 435/6; 435/7.2; 436/501

(57) ABSTRACT

A method for the determination and individual characterization of particles by means of at least two different detectable probes in a sample is proposed, wherein the particles, especially molecules or molecular aggregates, have at least one binding site, preferably a multitude of binding sites, for at least one of said at least two different detectable probes; said at least two different detectable probes are present in the sample; a measure of the number of bound probes and the mutual ratio of bound probes are established by determining particles; said determining being effected on the basis of single particles.

The present invention relates to a method for the determination and individual characterization of particles, especially subcellular particles, such as molecules, molecule aggregates or viruses.

One possible field of application of the present method, which has been realized in an exemplary manner, is the diagnosing of prion diseases and typing of different pathogenic strains. The prion diseases or transmissible spongiform encephalopathies are a group of transmissible neurodegenerative diseases in humans and animals, including Creutzfeldt-Jakob disease in humans as well as scrapie in sheep and BSE in cattle. Prion diseases are characterized by the deposition of an aggregated, pathological form of the prion protein (PrP) in the brain tissue of afflicted individuals, referred to as PrP^{Sc}. In principle, prion diseases are transmissible, and the transmissible agent is referred to as a “prion”. It is assumed that PrP^{Sc} is the critical or even the only component of the prion. A pathogen-associated nucleic acid could not be detected. The PrP^{Sc}, which is associated with disease and infectiosity, is distinguished from the form of the prion protein physiologically occurring in the organism (PrP^{c}) by its conformation, especially its high content of β-sheet structure, its relative resistance towards protease K and its tendency to form large multimeric aggregates. Within the scope of the so-called prion hypothesis, it is assumed that the PrP^{Sc} form can interact with the PrP^{c} form, thereby converting the endogenous PrP^{c} to the PrP^{Sc} form through a conformational change. Then, the thus newly formed PrP^{Sc} can itself interact with further PrP^{c} molecules and also convert them to PrP^{Sc}, so that large amounts of PrP^{Sc} can form from the endogenous PrP^{c} in an avalanche-like chain reaction.

An important phenomenon in prion diseases is the occurrence of different pathogenic strains. Even in passaging in hosts having an identical prion protein, e.g., mouse inbred strains, the pathogenic strains are constantly distinguished in various properties, such as incubation time, clinical symptoms, lesion patterns in the brain and transmissibility to other species. Within the scope of the prion hypothesis, the occurrence of different pathogenic strains in animals having the same PrP amino acid sequence means that different stable forms of PrP^{Sc} must exist, which can transform PrP^{c} into the respective pathological form. Also in the Creutzfeldt-Jakob disease of humans, various distinct subforms can be found which can be distinguished molecularly in a Western blot by a polymorphism in codon 129 of the prion protein gene (PRNP) and the size of the proteinase K resistant fragment of the prion protein, and are associated with different phenotypical manifestations of the disease.

It has been the object of the invention to provide a method by which individual pathological protein aggregates become ultrasensitively detectable in a homogeneous assay, and to characterize and type the detected aggregates.

In addition, this method should also be broadly applicable to detect and characterize other particles, preferably subcellular ones.

According to the invention, a method for the determination and individual characterization of particles by means of at least two different detectable probes in a sample is proposed, wherein
the particles, especially individual molecules or molecular aggregates, have at least one binding site, preferably a multitude of binding sites, for at least one of said at least two different detectable probes;
said at least two different detectable probes are present in the sample;
a measure of the number of bound probes and
the mutual ratio of bound probes are established by determining particles;
said determining being effected on the basis of single particles.

Further, according to the invention, a method is proposed for the characterization of pathological prion proteins by subspecies by labeling them with probe molecules, wherein the binding of at least two different probe molecules to the prion proteins is detected, and the subspecies is determined from the mutual ratio of quantities bound to different probe molecules.

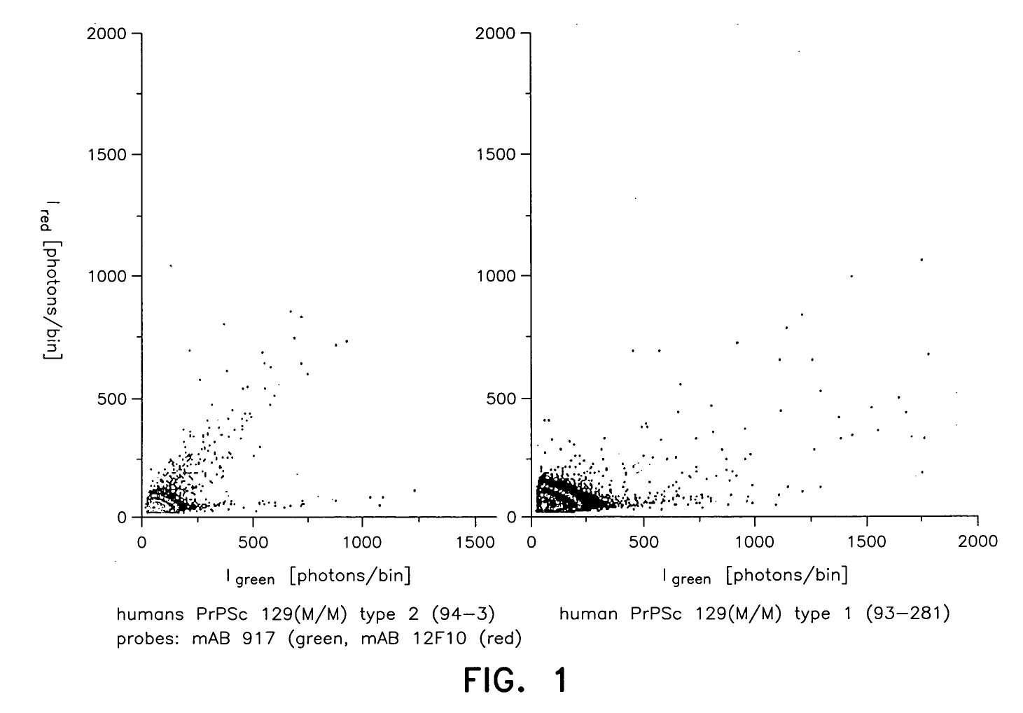

FIG. 1 shows dual-color intensity histograms of human PrP^{Sc} type 1 and type 2.

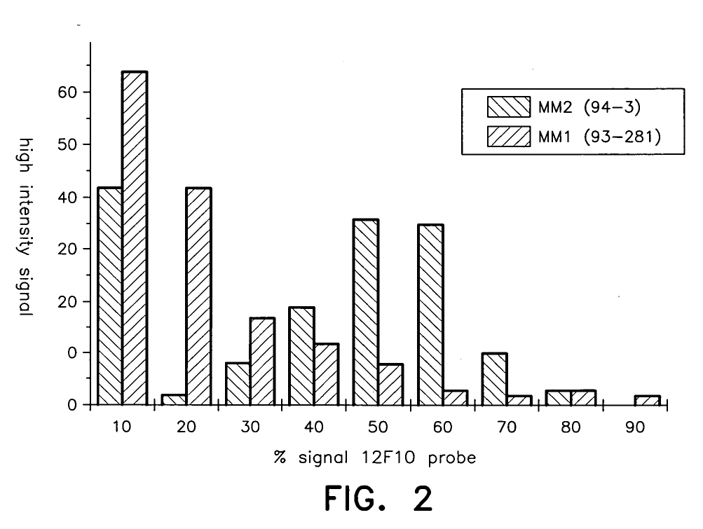

FIG. 2 shows the relative distribution of the signals of the bound PrP-specific probes (12F10-Cy5) and (pri917-Alexa488) for human PrP^{Sc}(129 M/M) type 1 and PrP^{Sc}(129 M/M) type 2. In the signal of MM 2 PrP, the proportions of the two probes are approximately equal while the signal of the MM 1 aggregates shows less than 20% red (12F10) signal.

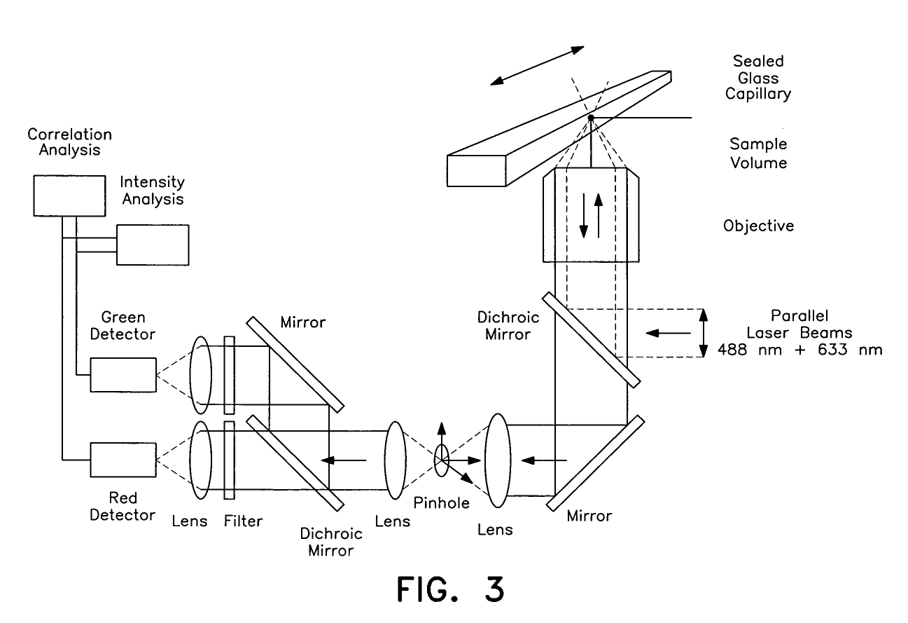

FIG. 3: Schematic set-up of the confocal dual-color fluorescence-spectroscopic apparatus.

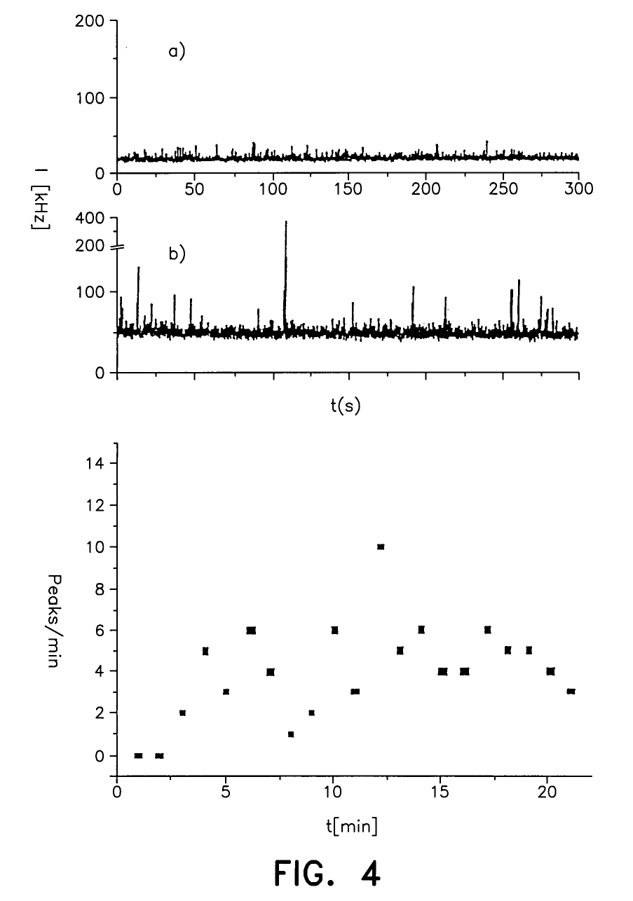

FIG. 4: Attachment of fluorescent probes to PrP aggregates. Trace of fluorescence intensity I a) in the absence and b) in the presence of pathogenic PrPSc aggregates in the cerebrospinal fluid. rPrP-Cy2 (c=10 nM) served as the probe, excitation at 488 nm, 180 μW, measuring time 21 min. Bottom: Number of detected aggregates per unit time in the course of the measurement.

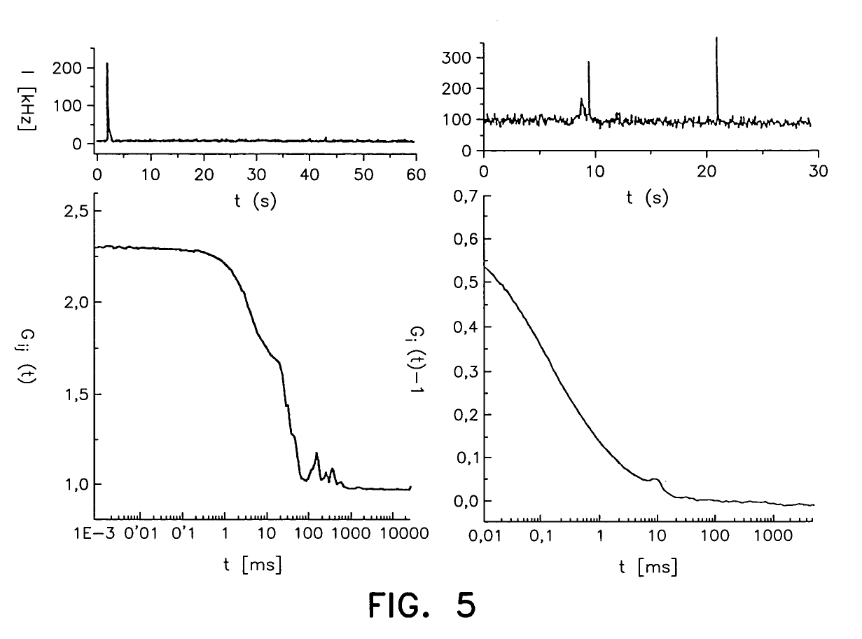

FIG. 5: Left: Determination of the aggregate size by a pair of heterologous probes. Preaggregated rPrP(90-231), monomeric concentration 0.1 μM, was detected by a pair of probes from rPrP-Oregon green (c=2 nM) and the antibody 15B3-Cy5 (c=10 nM). Total measuring time 20*1 min. During the individual measurements, only single labeled aggregates were detected, and their passing time determined by the cross-correlation signal. The fluorescence trace and cross-correlation signal of an individual measurement with τ≈15 ms are shown. Right: Homologous detection with rPrP-Oregon green.

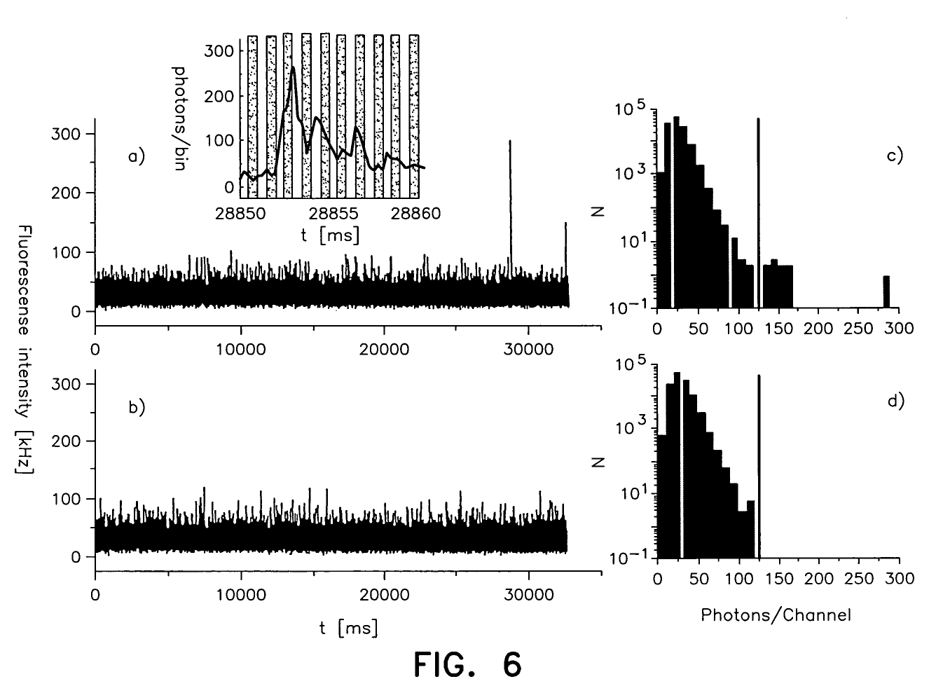

FIG. 6: Quantitative intensity analysis of the fluorescence signal. a) Fluorescence trace of probe+PrP aggregates; c) intensity histogram of a); b) fluorescence trace of the free probe (rPrP-Cy2); d) intensity histogram of the fluorescence signal b), bin width 250 μs.

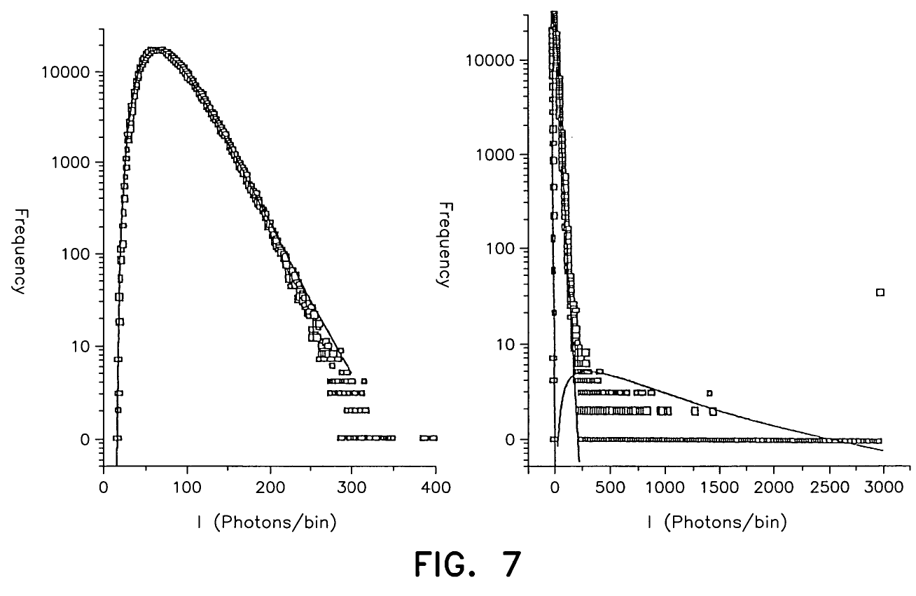

FIG. 7: Histogram of fluorescence intensity, bin width 500 μs. a) Antibody probe 3F4-Alexa488, c=6 nM, fitting by log normal distribution (see equation 9) with υ=32., σ=16. b) Prion rods, c=0.35 nM, fitting by a probe term with υ1=30 and a second term with υ2=200.

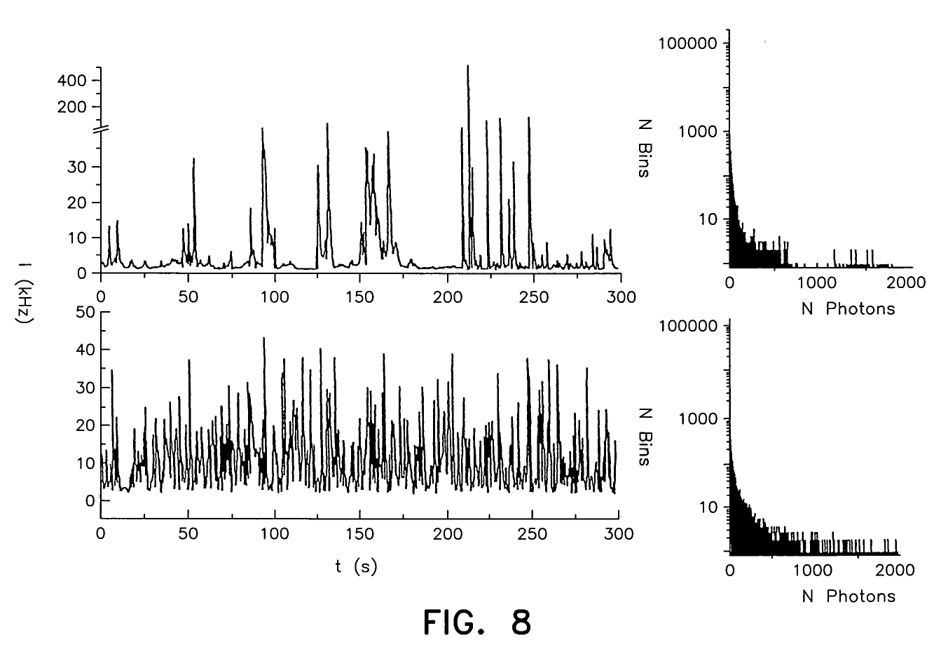

FIG. 8: Influence of sample movement on the number of detected events. Intensity trace and intensity histogram of fluorescent polystyrene beads in PBS+0.1% (w/v) NP40 upon excitation at 488 nm. Top: measurement without movement, bottom: with movement of measuring capillary by 1 mm/s. The number of detected events increases about 100 fold. Right: intensity histogram of the two measurements. By moving the sample, the number of channels with an intensity of >500 photons/channel is increased fourfold.

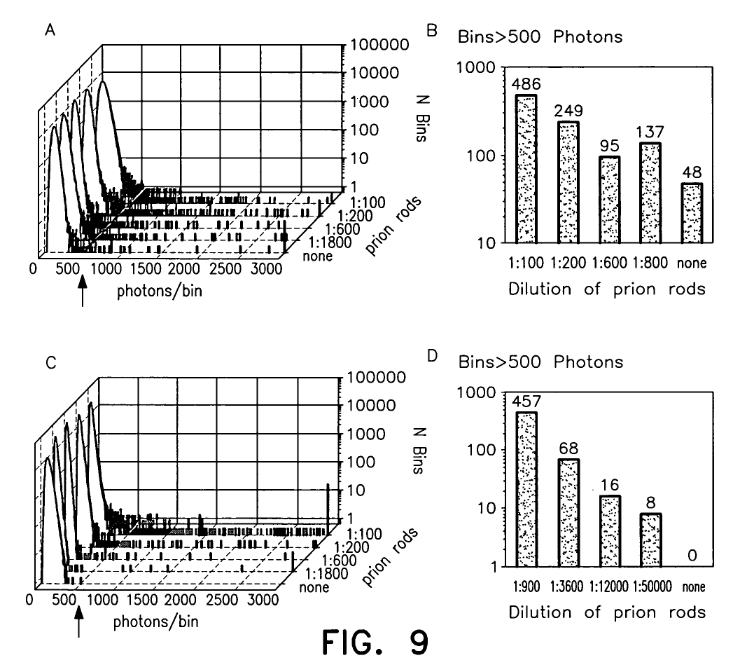

FIG. 9: Evaluation of different probe molecules. Hamster rPrP(90-231), labeled with Oregon green, (A,B) and monoclonal antibody 3F4, labeled with Alexa488, (C,D) were added to the cerebrospinal fluid of control patients to which prion rods had been added. The measurement is performed for 600 s with a sample movement of 1 mm/s and a bin width of 500 μs. The signal with high intensity was separated with a threshold (see arrow) of 500 photons/bin (B,D).

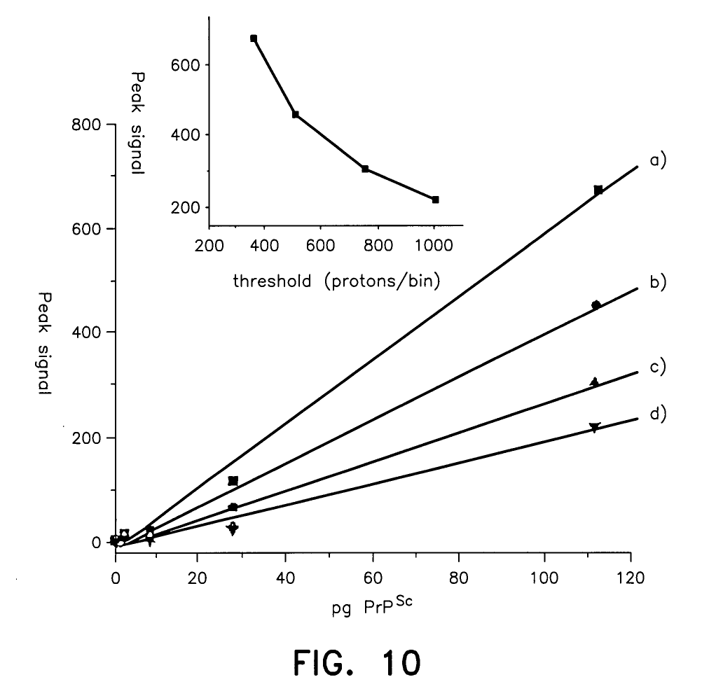

FIG. 10: Peak signal of the prion rods from FIG. 9 C as a function of the concentration of target molecules for a threshold of a) 350, b) 500, c) 750, d) 1000 photons/bin. The detection limit is 2 pg. Insert: Peak signal of 110 pg PrP^{Sc} as a function of the threshold value.

FIG. 11: Principle of two-channel intensity analysis. Antibodies labeled red and green (3F4-Alexa488, c=5 nM, 12F10-Cy5, c=6 nM) were added to the cerebrospinal fluid of control patients to which prion rods had been added (1:500). The measurement is performed for 600 s with a sample movement of 1 mm/s and a channel width of 500 μs. The high intensity coincident signal is separated from the signal of the free probes and the high intensity signal of the individual channels by a progressive threshold. Each dot corresponds to one intensity pair. The number of measuring channels is represented in a logarithmic plot on a color scale.

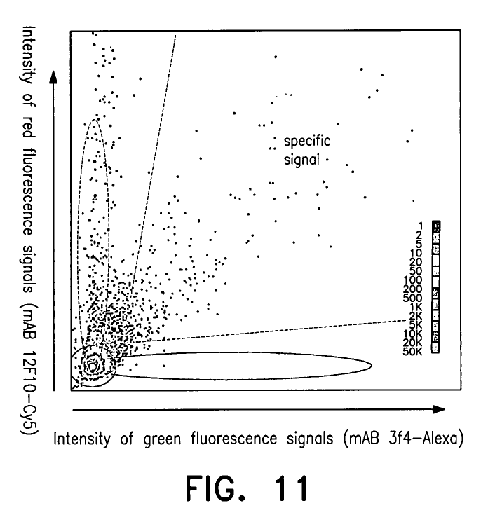

FIG. 12: Specificity of detection of Aβ and PrP target molecules by two-channel SIFT. Specific and non-specific pairs of probes and target molecules were combined: a) preaggregated Aβ(1-42) peptide (1 μM)+Aβ antibody (6E10-Cy5, p42-Alexa), b) Aβ(1-42) peptide (1 μM)+PrP antibody (3F4-Alexa, 12F10-Cy5), c) prion rods 1:1000+PrP antibody (3F4-Alexa, 12F10-Cy5), d) prion rods 1:1000+ irrelevant antibodies (anti-IL8-Oregon green, anti-Aβ-Cy5).

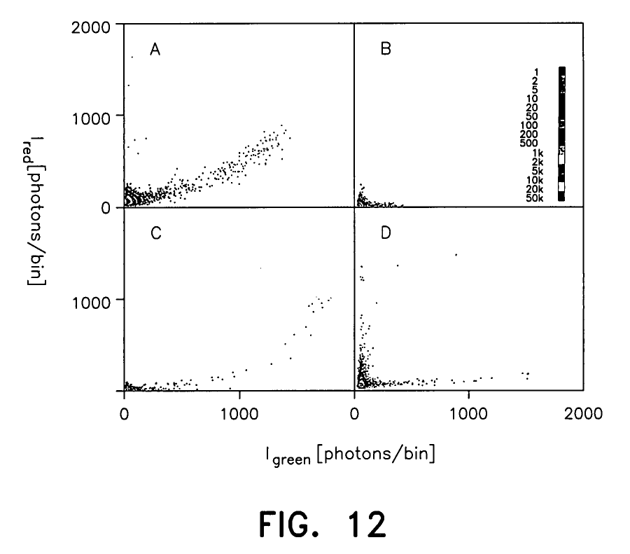

FIG. 13: Western blot and two-channel SIFT measurement of a dilution of prion rods in cerebrospinal fluid. The brain homogenizate of a scrapie-infected hamster 263 (a-f) and prion rods in cerebrospinal fluid were diluted as stated. A: PrP^{Sc} was digested with proteinase K (100 μg/ml) for 30 min at 37° C., followed by detection by Western blot with antibody 3F4. B: In a parallel measurement, aliquots of the prion rods were measured by two-channel SIFT, and the signal evaluated as described in FIG. 11.

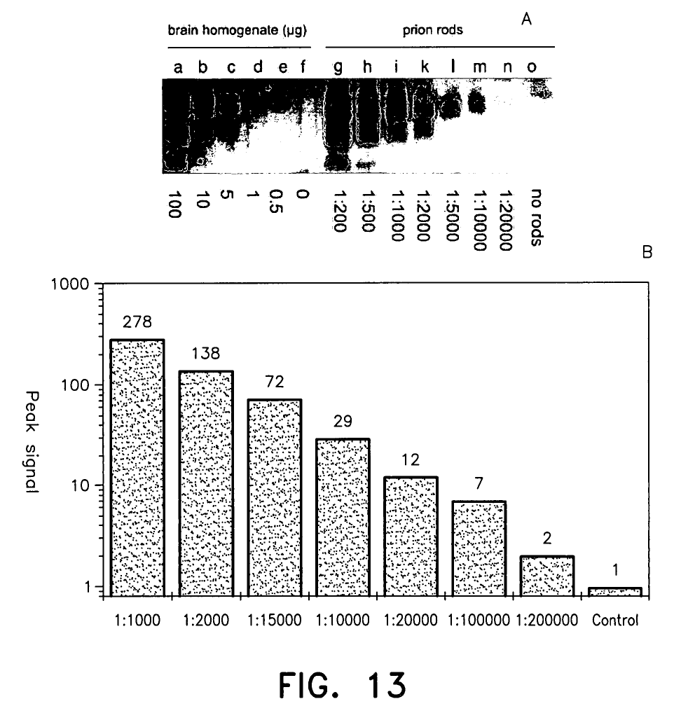

FIG. 14: Histogram representation of the two-channel SIFT measurement of a dilution of prion rods in cerebrospinal fluid. A: dilution 1:2000, B: 1:10⁵, C: without PrP^{Sc}.

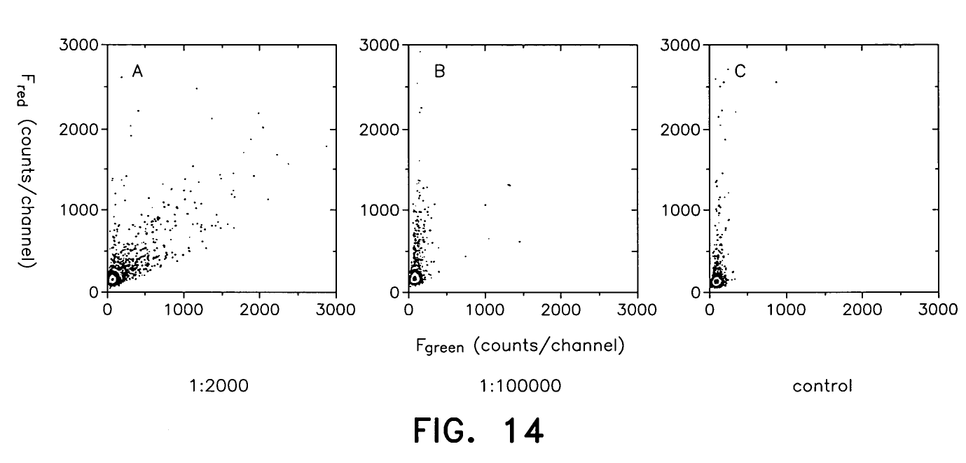

FIG. 15: a) Cross-correlation signal of a dilution of prion rods in cerebrospinal fluid. PrP^{Sc} concentration: 160 pM (solid line), 56 pM (dashes), 20 pM (dots), 6 pM (dots and dashes), 2 pM (short dashes), without rods (thin solid line). b) Plot of cross-correlation amplitude Gᵢⱼ(0) against the amount of PrP^{Sc} employed. c) Plot of the number of measuring channels of high fluorescence intensity of two-channel SIFT analysis against the cross-correlation amplitude Gᵢⱼ(0) of aggregated Aβ(1-42) peptide in different media. The measurement was performed as in FIG. 11 in cerebrospinal fluid (CSF) and buffer with and without detergent (buffer: PBS, PBS+0.1% NP40, RIPA, PBS+0.2% SDS, CSF, CSF+0.1% NP40). pAB42-Alexa and 6E10-Cy5, c=6 nM, serve as specific antibody probes. Both signals were proportional independently of the medium employed.

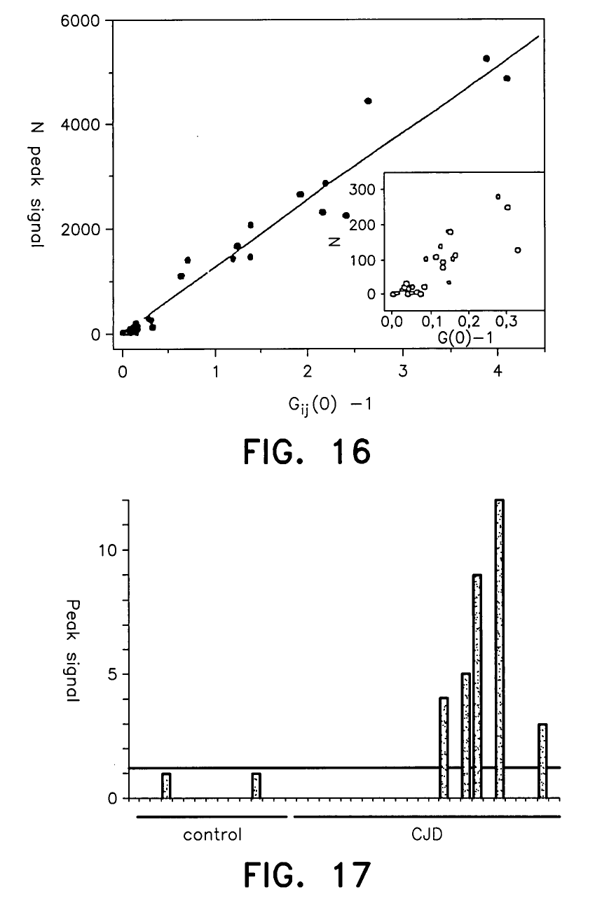

FIG. 17: Two-channel SIFT measurement in cerebrospinal fluid samples of CJD and control patients. The measurement was performed for 600 s with a channel width of 0.5 ms as described in FIG. 11. The monoclonal antibodies 3F4-Alexa488 and 12F10-Cy5 served as probes. In 5 out of 24 CJD cerebrospinal fluid samples, the signal was above a threshold of one channel, whereas none of the controls with other neurodegenerative diseases contained a positive signal.

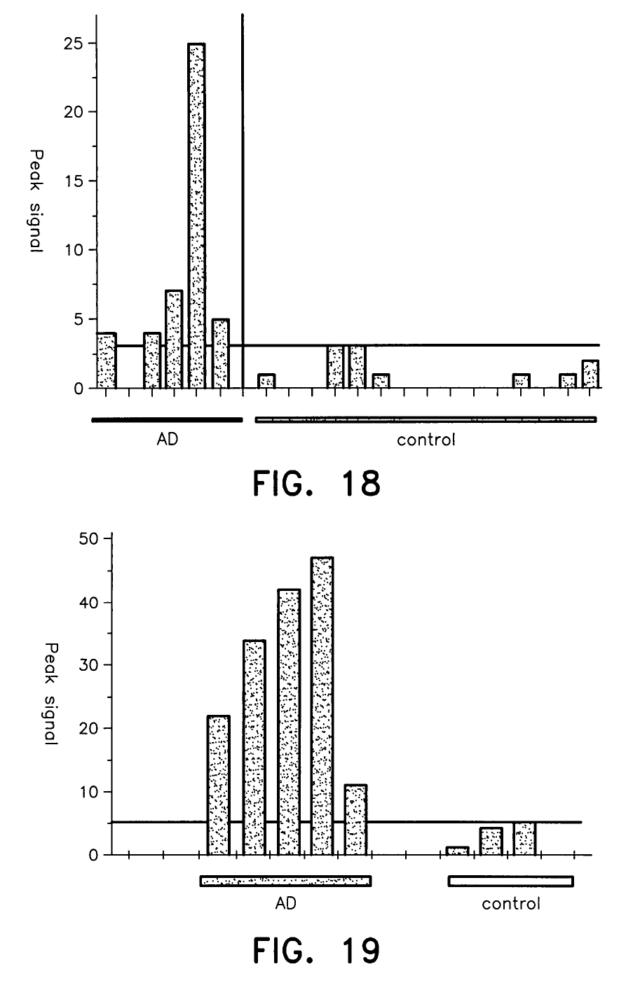

FIG. 18: Two-channel SIFT measurement in cerebrospinal fluid samples of AD and control patients. The measurement was performed for 600 s with a channel width of 0.5 ms as described in FIG. 11. The antibodies pAB42-Alexa488 and 6E10-Cy5 served as probes. In 5 out of 6 patients with clinical Alzheimer's diagnosis, but in none of the controls, the signal was above the set threshold value.

FIG. 19: Two-channel SIFT measurement in cerebrospinal fluid samples of AD and control patients. The measurement was performed for 600 s with a bin width of 0.5 ms as described in FIG. 11. The antibodies pAB42-Alexa488 and 6E10-Cy5 served as probes. Immediately after removal, the samples were put in safekeeping.

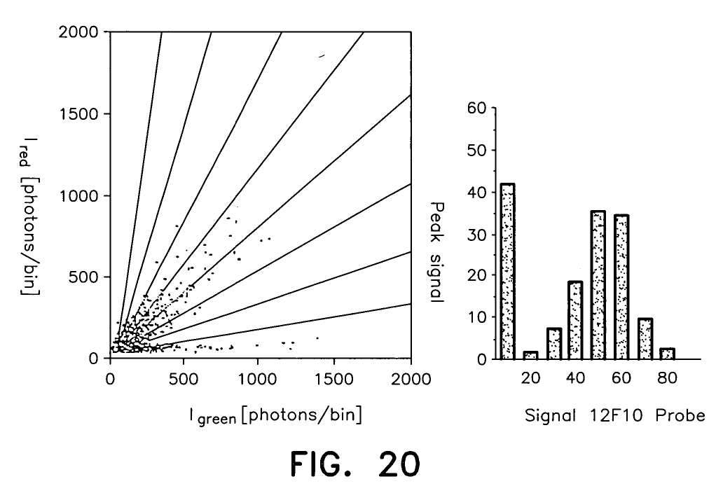

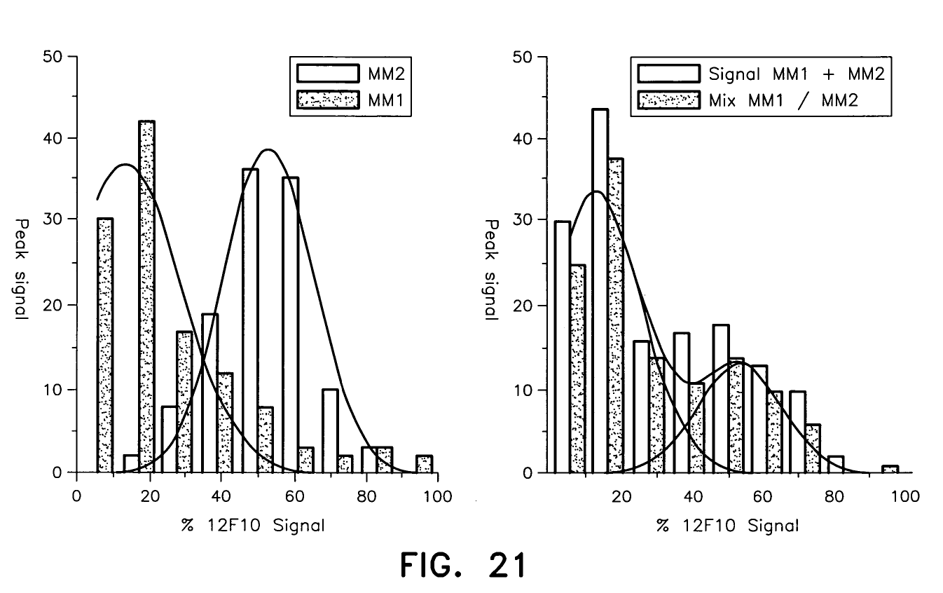

FIG. 20: Determination of the binding ratio of the samples. Signal of a two-channel SIFT measurement on human PrP^{Sc} (129M/M) type II. The measurement was performed for 600 s with a channel width of 0.5 ms as described in FIG. 11. The antibodies Pri917-Alexa488 and 12F10-Cy5 served as probes. The PrP^{Sc}-specific signal of >8Iₘₐₓ was summed up in nine sectors of identical signal ratios.

FIG. 21: PrP^{Sc} type I and type II in a SIFT signal of human PrP^{Sc}(129 M/M). The purified PrP^{Sc} was diluted 1:10 in cerebrospinal fluid of control patients to which 0.1% NP40 was added. The measurement and evaluation was as described in FIG. 20. a) Signal proportions of the two probes in the measurement of (M/M) type I and (M/M) type II PrP^{Sc} with fitting by a normal distribution. b) Signal proportions of the two probes in the summing up of individual measurements of PrP^{Sc} type I and type II (gray), and measurement of a mixture of the two PrP^{Sc} types.

In the method according to the invention, the mutual ratio of bound probes is preferably established by determining particles in a measuring volume which is a subvolume of the sample to be examined. Particularly preferred is determination on the basis of single particles which are within the measuring volume at different times.

The detection of different bound probes is preferably effected simultaneously on one particle.

Preferably, the measuring volume is ≦10⁻¹²l, especially ≦10⁻¹⁴l. The measurement is performed, in particular, using a confocal microscopic set-up, a near-field set-up or a set-up for multiphoton excitation. The determination and characterization of particles is performed, in particular, in a homogeneous assay method without washing steps.

One advantageous possibility of characterizing particles, such as pathological prion protein aggregates (referred to as “target molecules” in the following), is labeling with suitable fluorescence-labeled probe molecules, followed by the detection and analysis of individual aggregates. This is accomplished by a measuring method based on an implement set-up for dual-color fluorescence spectroscopy, hereinafter referred to as SIFT (scanning for intensely fluorescent targets) in a specific embodiment. The method according to the invention is based on a time-resolved intensity analysis of a fluorescent signal from an open volume element defined by a confocal figure of one or more excitations lasers concentrated in one focus. This method is distinguished from the prior art of FCS-based amyloid aggregate detection (Pitschke et al., 1998) especially by the following modifications:
a) According to the invention, the quantification of the particle-caused signal fraction is preferably effected by analyzing the intensity distribution of a measured detection signal, especially a fluorescence signal, in successive time windows with detection times of constant or variable lengths in the range of micro- to milliseconds, whereby the very intensive signal of the multiply labeled target molecules can be separated from the background signal of free probe molecules. Alternatively, the intensity-based separation of the signal fraction caused by the target molecule could also be effected by an algorithm for peak detection and analysis.
b) According to the invention, the sample is preferably subjected to scanning by producing an essentially constant relative movement between the sample and the measuring volume. In a preferred measuring set-up, this goal is achieved by a meandering movement of the sample filled into a measuring capillary. In a further embodiment, this aspect may also be realized by a lens system which allows for movement of the focus, or by a flow capillary. The scanning brings about two advantages:
   1. The volume examined and thus the measuring sensitivity is significantly increased.
   2. For large, very slowly diffusing target molecules, the average dwelling time in the focus is no longer determined by the diffusion time T_{diff}, but by the scanning speed. This is advantageous because substantially all target molecules are mapped on about the same number of measuring channels. Thus, the number of very intensive channels becomes a direct measure of the number and concentration of highly labeled target molecules.
c) According to the invention, the use of antibodies as probe molecules is preferred. As compared with monomeric aggregate components, these have the advantage of little self-aggregation. Although this point is preferred according to the invention, the method according to the invention can be performed, in principle, with any probe which specifically binds to the target molecule, preferably fluorescence-labeled ones.
d) In particular, the method according to the invention employs a simultaneous analysis of two or more probes, especially fluorescent probes, which are separately measurable in the same measuring volume and emitting in different wavelength regions or polarization planes. Preferably, the data acquired according to the invention are established from multiple, especially dual, color or polarization measurements and arranged in a corresponding multidimensional, especially two-dimensional, array for evaluation, for example, represented as an intensity histogram. The number of channels having simultaneously high values for the several or two colors/polarizations is a measure of the number and concentration and specific for target molecules labeled with several, especially two, independent probes. As already mentioned under item a), alternatively, a multicolor peak analysis is also possible.

According to the invention, pathological protein aggregates can be detected as particles, especially prion proteins by subspecies, by labeling with probe molecules.

Preferably, the binding of at least two different probe molecules to the particles forming the protein aggregates is detected, and the subspecies is determined from the mutual ratio of amounts of different bound probe molecules.

The method according to the invention may also be used for pathogenic strain typing or for examining the relative binding of proteins from different species to pathological protein aggregates of a particular species for estimating an interspecific barrier for the transmission of a disease.

In a further embodiment of the method according to the invention, it can serve for the examination of degenerative diseases, especially neurodegenerative diseases, with formation of pathological aggregates, especially aggregates which contain prion protein, APP, Tau, synuclein or proteins having a polyglutamine sequence, such as huntingtin, or fragments or derivatives of such proteins as a component.

In particular, the method according to the invention is suitable for examining subcellular particles, especially including the phenotypical analysis of viral particles, or for analyzing nucleic acids using antisense probes.

In addition to the increased specificity in the detection of target molecules, the method according to the invention has an additional potential:

For essentially every detected target molecule, the relative labeling intensity of the probes of different colors can be measured separately. In contrast to the absolute intensity of the individual colors, this labeling ratio is essentially independent of the route which the respective target molecule takes through the detection volume for the different separately detected colors when the volume elements are almost congruent. Thus, the simultaneous measurement of several different probes on one single particle can be considered an internal standard on the level of the individual particles by relating the measured values to one another. Therefore, for a homogeneous population of target molecules, the labeling ratio for all detected particles is similar, and therefore, in a two-dimensional intensity histogram, the target molecule will scatter specific signals around a straight line whose steepness is determined by the relative binding of the two probe molecules analyzed. When a different type of target molecule having differing binding properties is analyzed, a correspondingly different labeling ratio results (FIG. 1). Thus, the relative binding of two different probes can be determined easily and quickly in a homogeneous assay under defined buffer conditions and, under suitable conditions, respectively yields a characteristic value for different types of target molecules.

In the case of the prion diseases, due to the occurrence of different pathogenic strains distinguishable by their biological behavior, it is to be considered even in hosts having identical PrP primary structures that different pathological forms of PrP^{Sc} exist which are evidently distinct only by their conformation or aggregate structure. By a different antibody binding depending on conformation, these different forms or prion strains should be basically distinguishable when suitable monoclonal antibodies are available. Thus, when purified PrP^{Sc} aggregates from Creutzfeldt-Jakob patients are examined, a different binding behavior of monoclonal antibodies 12F10 and Pri917 is found depending on whether the pathological prion protein is of type I or type II. Both in humans and in the animal kingdom, the typing of pathogenic strains is of great epidemiological importance. Of particular relevance is the identification of the BSE pathogenic strain after transmission to other species. Especially in humans, pathogen typing should be additionally important for prognosis and perhaps therapy.

The typing through the relative binding of different probe molecules using the method according to the invention has several conceptional advantages:
1) Since every target molecule is analyzed separately, in principle, mixtures of different target molecules may also be examined and the quantitative ratio of the components determined. This is a fundamental difference between the method proposed according to the invention and all the methods in which measured values resulting from the integration or averaging of measuring values across ensembles of target molecules are obtained.
2) For typing, probes having moderately differing affinities for the different types of target molecules are sufficient, all-or-none binding is not required.
3) From the accessibility of different epitopes recognized by different probe molecules in different types/pathogenic strains, conclusions on the three-dimensional structure can be drawn.
4) Since the relative binding of the different probe molecules is determined by simultaneous measurement of these probe molecules on individual target molecules (particles), this binding ratio is also influenced by the interaction of such probe molecules (e.g., steric competition). Therefore, the simultaneous measurement in the presence of the different probes which can be separately detected yields more information than would be yielded by the separate determination at different times, in different measuring volumes or in separate measuring samples.
5) Small amounts of target molecule in low concentrations are sufficient, and previous purification is not necessary, so that the target molecule can also be analyzed under almost native physiological conditions.
6) As compared to established methods of PrP^{Sc} typing (Western blot), the present method allows a quick test in a homogeneous assay so that a large number of samples can be analyzed (diagnostic screening or screening for active substances).

The method according to the invention is not basically limited to the above described concrete application in the field of typing of different prion strains. In principle, it is possible to analyze a wide variety of preferably subcellular particles which can be labeled with probes, especially fluorescence-labeled probes. The above stated advantages apply here as well, mutatis mutandis. In particular, the following fields of application may be mentioned:
a) In the field of prion diseases, in addition to pathogenic strain typing, the relative binding of PrPC from different species to prion protein aggregates from a particular species can be examined, which enables the respective interspecific barrier for disease transmission to be estimated.
b) Other (neuro)degenerative diseases with formation of pathological aggregates, such as Alzheimer's disease, in particular, can be examined analogously. In this case too, subtypes of pathological aggregates having potentially different diagnostic, prognostic and therapeutic significance can be recognized. In particular, there may be mentioned the analysis of aggregates which contain prion protein, APP, Tau, synuclein or proteins having a polyglutamine sequence, such as huntingtin, or fragments or derivatives (e.g., phosphorylated or glycosylated derivatives) of such proteins as at least one component.
c) Other subcellular particles can be examined analogously, up to the phenotypical analysis of viral particles.

The attachment of several probes to one pathological aggregate can be used for the detection of individual aggregates of, more generally, target molecules in solution. The development of this principle to a highly sensitive detection method and its exemplary application in the diagnostics of cerebrospinal fluid in Creutzfeldt-Jakob disease (CJD) and Alzheimer's disease are set forth in some detail below.

Theoretical Basics

Correlation Analysis of Several Components

If several fluorescent components i coexist in a solution, they contribute proportionally to the correlation function [15]. In the case where the components of the solution have different quantum efficiencies of fluorescence, i.e., shine with different “brightnesses”, the different detection probabilities of the molecules should be considered. Therefore, a relative quantum yield αᵢ≡Qᵢ/Q₁ is defined. Then, the correlation function reads thus [27]:

$$G ( τ ) = \frac{1}{{N}_{ges}} {\sum }_{i = 1}^{n} {x}_{i} {α}_{1}^{2} {diff}_{i} \\ where \\ {N}_{ges} \sum {N}_{i} ; \\ {x}_{i} = {C}_{i} / \sum {C}_{i} \\ {diff}_{i} ≡ {( 1 + \frac{τ}{{τ}_{D , i}} )}^{- 1} {( 1 + \frac{{ω}_{0}^{2}}{{z}_{0}^{2}} \frac{τ}{{τ}_{D , i}} )}^{- 1 / 2} \qquad ( 1 )$$

and Cᵢ is the concentration of component i. It is to be noted that highly fluorescent molecules are overrepresented as compared with their proportional concentration due to the fact that the square of αᵢ, is found in the correlation function. The effective luminosity of a molecule which bears a lot of fluorophors is very much higher than that of molecules which bear only one fluorophor. Therefore, in the case of aggregation, the passage of a single highly labeled aggregate through the focus can completely dominate the correlation curve.
Dual-Color Cross-Correlation Analysis

In the analysis of an auto-correlation signal, there is often a problem of superposition of many dynamical processes, e.g., by different diffusing molecular species. If the molecules are not substantially different in size or if more than two components are present in solution, the signal fractions of the individual components can no longer be determined with certainty [25].

A solution to this problem is offered by the technique of dual-color cross-correlation analysis developed in [3] and worked-out by Petra Schwille both theoretically and experimentally. The technique has been described in detail in [23] and [24]. In the measurement, the fluctuation in the signal of two fluorophors whose emission spectra overlap as little as possible is examined. If two molecular species are labeled with these dyes, the interaction of the labeled molecules can be followed by cross-correlating the fluctuation of the two fluorescence signals. Also, similar molecules can be provided with different labels for characterizing their interaction with one another or with a third partner. When the molecular species i and j bind to one another or to a common interaction partner, a molecular species ij is formed which bears both fluorophors. This is the only component to contribute to the cross-correlation signal. It was used as a reference in the detection of pathological aggregates. The fluorescence signal Fᵢ(t) is compared with Fⱼ(t+τ) in the same measuring volume. Then, the following holds for the scaled cross-correlation signal Gᵢⱼ(τ):

$${G}_{ij} ( τ ) = \frac{〈 δ {F}_{i} ( t ) δ {F}_{j} ( t + τ ) 〉}{〈 {F}_{i} ( t ) 〉 〈 {F}_{j} ( t ) 〉} \qquad ( 2 )$$

For a color i, by analogy with equation (7), the fluctuation of the fluorescence signal results from the sum of fluctuations of all molecules which bear the fluorophor i. If the molecular species nᵢ have different relative brightnesses, the emission characteristics Wₙ(\vec{r}) with respect to the respective excitation colors should again be considered:

$$δ {F}_{i} ( t ) = {\sum }_{n} {\int }_{V} {W}_{{n}_{i}} ( \vec{r} ) δ {C}_{n} ( \vec{r} , t ) ⅆ \vec{r} \qquad ( 3 )$$

In the case of an aggregation process, the system contains many different molecular species which bear different numbers of fluorophors and whose quantum yields can again be reduced to different extents by the different molecular environments. The fluctuation term can become very complex due to the high number of different emission characteristics W(\vec{r}). In the case of small aggregates, if it is assumed that the aggregation process does not substantially alter the emission of the dyes, then the denominator of equation 2 remains constant, which yields:

$${G}_{ij} = const \frac{{\sum }_{m} {\sum }_{n} mn 〈 {N}_{mn} 〉 {diff}_{mn}}{〈 {N}_{i , 0} 〉 〈 {N}_{j , 0} 〉} \qquad ( 4 )$$

wherein diff is defined as in equation (1). Nₘₙ is the number of aggregates containing m monomers of species i and n monomers of species j, and N_{i,o} and N_{j,o} are the numbers of free monomers i and j, respectively, in the measuring volume at the beginning of the experiment. In the case of heterogeneous aggregates, even this expression is still too complex to allow a quantitative evaluation.

In the case of a simple dimerization, which should be the initial step of each aggregation process at low concentrations, the expression Gᵢⱼ is reduced to a single diffusion component with the time constant τᵢⱼ:
Gᵢⱼ(τ)=const<Nᵢⱼ>di∫∫ᵢⱼ Gᵢⱼ(0)α<Nᵢⱼ> (5)

Thus, a linear relation is obtained between the inverse correlation amplitude G(τ)⁻¹ and τ:

$${G ( τ )}^{- 1} = \frac{N}{{τ}_{ij}} \cdot τ + N \qquad ( 6 )$$

From equations (4) and (5), several features of the cross-correlation function which make it attractive for the examination of binding processes are immediately evident:
1. Only the diffusion component of the doubly labeled molecules appears in the denominator of Gᵢⱼ. Also in mixtures with monomers and homo-multimers, it can be characterized isolatedly.
2. Under the precondition that the total fluorescence does not change in the course of the reaction, the amplitude G(0) is directly proportional to the concentration of the doubly labeled molecules. Thus, the kinetics of a binding process or cleavage process can be followed in a simple way [24], [6].
3. Since the transitions of two fluorescence photons into triplet state is independent even when they are bound to the same molecule, the cross-correlation signal does not contain a triplet contribution [24]. This enables fitting of the measured values having a lower number of free parameters and thus a good matching even for a poorer signal-to-noise ratio.
   Intensity Distribution of the Fluorescence Signal

Another parameter which can be used for the characterization of a molecule in addition to the diffusion time is the specific brightness of the molecule. An analysis of fluorescence intensity based on higher modes of the correlation function was performed by Qian in 1990 [18]. A good experimental measure of the specific brightness is the count rate of fluorescence photons per molecules (cpms). For a constant excitation, this quantity is proportional to the product Q of the fluorescence quantum yield and the absorption cross-section of the molecule [5]. Thus, it is characteristic of the molecule. In the case of an aggregation process or also of the detection of aggregates already present in the solution, the binding of many monomers with identical fluorophors produces the greater brightness of the aggregate. Not considering quenching and eclipsing effects which can reduce the quantum yield of the fluorophors in the aggregate, the relative brightness would then be proportional to the number of bound fluorophors. In practice, however, this consideration only allows a very coarse estimation of the number of bound fluorophors.

The intensity distribution of the fluorescence photons could be calculated if the detection function W(\vec{r}) of the molecule was known. According to equation (7), it is represented by
W(\vec{r})=Iₐ(\vec{r})CEF(\vec{r})Q (7)
where Iₐ(r) is the excitation profile and CEF(r) is the collecting function of the optical set-up. For an analytical solution of the correlation function, W(r) was approximated by a three-dimensional Gaussian profile [21]. However, the intensity distribution of the fluorescence photons shows significant deviations from this approximation [5]. If a known detection function W(\vec{r}) is assumed, the distribution of the fluorescence photons within an infinitesimal volume dVᵢ having a constant detection function Wᵢ can be calculated. It is the product of two Poisson distributions, i.e., the distribution of the number N of molecules within the volume dVᵢ and the distribution of the number of photons n detected from a molecule in volume dVᵢ [5].

$${P}_{i} ( n ) = {\sum }_{N = 0}^{∞} N \frac{{〈 N 〉}^{N}}{N !} {c}^{- 〈 N 〉} \cdot \frac{{〈 n 〉}^{n}}{n !} {c}^{- 〈 n 〉} \qquad ( 8 )$$

where <N>=∫CdVᵢ and <n>=QWᵢT;
wherein C is the concentration of the molecules and T is the bin width, i.e., the length of the time intervals in which photons are summed up. This approach is based on two assumptions:
1. The molecules are static, and therefore it must hold that T<<τ_{D}
2. The detection function of a molecule is the product of a general detection function W(\vec{r}) and a molecule-specific constant Q. In aggregate detection, the first assumption is met only for the case of stationary measurement. Otherwise, a longer bin width was chosen in order to maximize the ratio between the aggregate signal and the probe background. The second assumption is only conditionally met in the case of single chromophores, since the transition to the radiationless triplet state reduces the quantum yield. This transition depends on the excitation intensity and thus on the excitation profile. However, this limitation can be neglected for multichromophorous molecules.

When an exact characterization of the intensity distribution is not required, but merely a very intensively fluorescent component is to be separated by a threshold value, the distribution of the detected photons per bin, n, can be fitted empirically by a “skew” normal distribution. A log normal distribution may serve as a fitting model.

$$P ( n ) = \frac{1}{n \sqrt{2 π {σ}^{2}}} {ⅇ}^{- {( log n - v )}^{2} / 2 {σ}^{2}} \qquad ( 9 )$$

where υ represents the expected value and σ represents the standard deviation of the distribution.
Materials

The sources of acquisition of the chemicals, chromatographic materials and proteins employed are stated below. All chemicals employed were of the highest purity grade available.

| Supplier | Material |
|---|---|
| Calbiochem, Nottingham (UK) | NP-40 Detergent 10% |
| MoBiTec, Gottingen (Germany) | Microspin columns |
| Pharmacia, Freiburg (Germany) | Sephadex G 75 |
| | Sephadex G 15 |
| Sigma, St. Louis, MO (USA) | Dulbecco's PBS |
| | SDS |
| | Tween20 |
| VitroCom Inc., Mountain Lakes, | Glass capillaries |
| NJ (USA) | (50 × 2.6 × 0.2 mm) |
| Wiederholdt & Hutter (Germany) | Deutscher Wappenlack |

Fluorescent dyes

| | |
|---|---|
| Amersham, Arlington Heights, IL, | Cy5 Labeling Kit |
| (USA) | |
| Molecular Probes, Eugene, OR (USA) | Alexa Fluor 488 Labeling Kit |
| | FluoSpheres (505/515) |
| | Oregon Green 488 Labeling Kit |

Proteins

| | |
|---|---|
| Bachem AG (Germany) | Amyloid β protein (1-42) |
| Sigma, St. Louis, MO (USA) | Proteinase K |
| | Bovine serum albumin (BSA) |

Buffers and Stock Solutions

The following list contains all buffers and stock solutions employed, the stock solutions having been prepared with demineralized water, and their abbreviations. Buffer solutions were sterile-filtered through a membrane filter (0.22 μm, Millipore) prior to use.

| | |
|---|---|
| Abbreviation | Composition |

| | | | | |
|---|---|---|---|---|
| AP | SDS | | 3% | (w/v) |
| | Tris-HCl | pH 6.8 | 60 | mM |
| AS | NaCl | | 100 | mM |
| | KAc/HAc | pH 5.0 | 10 | mM |
| B buffer | Na₂HPO₄/NaH₂PO₄ | pH 7.0 | 20 | mM |
| Er buffer | Glycine-HCl | pH 2.7 | 100 | mM |
| N buffer | Tris-HCl | pH 7.0 | 1 | M |
| NaPi | Na₂HPO₄/NaH₂PO₄ | pH 7.2 | 10 | mM |
| NaP2 | Na₂HPO₄ | pH 8.4 | 0.5 | M |
| NaC | Na₂CO₃ | pH 9.2 | 1 | M |
| Lysis buffer | NaCl | | 100 | mM |
| | EDTA | | 10 | mM |
| | NP-40 | | 0.5% | (v/v) |
| | Na deoxycholate | | 0.5% | (w/v) |
| | Tris-HCl | pH 7.4 | 10 | mM |
| PBS | NaCl | | 100 | mM |
| | Na₂HPO₄/NaH₂PO₄ | pH 7.2 | 10 | mM |
| PBSN | NaCl | | 100 | mM |
| | Na₂HPO₄/NaH₂PO₄ | pH 7.2 | 10 | mM |
| | NP-40 | | 0.1% | (w/v) |
| PBSS | NaCl | | 100 | mM |
| | Na₂HPO₄/NaH₂PO₄ | pH 7.2 | 10 | mM |
| | SDS | | 0.2% | (w/v) |
| RIPA | NaCl | | 100 | mM |
| | EDTA | | 1 | mM |
| | NP-40 | | 1% | (v/v) |
| | Na deoxycholate | | 0.5% | (w/v) |
| | Tris-HCl | pH 7.4 | 10 | mM |

Prion Protein

rPRP: As a model system for the examination of the aggregation of the prion protein, a recombinant prion protein produced in E. coli which was homologous to amino acids 90-231 of the prion protein from Syrian hamster was predominantly employed. Thus, it corresponded to the protease-resistant core of pathological PrPII, was different from the natural protein, but not glycosylated, and did not have a membrane anchor. Otherwise, its structure corresponded to amino acids 90-231. The protein was expressed in a STII TIR vector in E. coli strain 27C7 as described by Mehlhorn [13]. The protein was available as a stock solution with a concentration of 1 mg/ml in PBS+0.2% SDS (w/v).

Prion rods: The preparation of aggregated PrP (27-30) from Syrian hamsters, the so-called prion rods, is described in [17]. The protein was in a sonicated state in a concentration of 30 μg/ml in NaPi+0.2% SDS (w/v).

Human PrP^{Sc}: The different subtypes of Creutzfeldt-Jakob disease were differentiated by Parchi et al. (1997) using this material by conventional methods by means of strain types (1/2) and the polymorphism at codon 129 of the human prior protein. The same material was used for direct differentiation of PrP^{Sc} types I and II by SIFT measurement.

Antibodies

Within the scope of this work, different specific antibodies against epitopes of the prion protein and Aβ(1-42) peptide were used. They are stated below.

Antibody Pri917 is directed against amino acids (214-230) of human PrP.

Antibody 3F4 is directed against amino acids (109-112) of hamster PrP and has a somewhat weaker affinity for human PrP. It was prepared according to [7].

Antibody 15B3 specifically recognizes the aggregated PrP^{Sc} isoform. It was prepared by Prionics (Switzerland).

Antibody 12F10 is directed against amino acids (142-160) of human PrP (Krasemann [8]). It was supplied by IBA, Heiligenstadt (Germany).

The A/3-specific antibody 6E10 is directed against the N terminus of the A β peptides (1-17).

Antibodies employed are stated in the following:

| Designation | Antigen | Supplier |
|---|---|---|
| MAMI (IgM) | β-Amyloid(1-40) | BMA Biomedicals, |
| | | Augst (CH) |
| Anti-IL8 (IgGI) | human IL-8 | Sigma BioSciences, |
| | | St. Louis, MO (USA) |
| Anti-A40 (serum) | β-Amyloid 1-40 | Sigma BioSciences, |
| | | St. Louis, MO (USA) |
| pAB42 (polyclonal AB) | β-Amyloid 1-42 | Oncogene Cambridge MA |
| | | (USA) |

Cerebrospinal Fluid Samples

For the diagnostic examination, cerebrospinal fluid from patients was used [30, 31]. Withdrawal within the scope of the study was performed with the approval of the patients.

For CJD diagnostics, cerebrospinal fluid from 37 patients afflicted with neurodegenerative diseases was used. These included 11 neuropathologically ascertained cases, and 13 cases where the diagnosis was considered probable by epidemiological criteria [28].

For Alzheimer's diagnostics, cerebrospinal fluid from 6 patients where the diagnosis of Alzheimer's disease was ascertained by biochemical (concentration Aβ42/40/38), neurological and psychological criteria, and from 12 control patients. The cerebrospinal fluid samples were obtained within the scope of neurological routine diagnostics. The samples of the clinical studies are not standardized with respect to pretreatment. After withdrawal, they were stored at −70° C. and repeatedly thawed for biochemical examinations.

Cerebrospinal fluid from 5 AD patients and 4 control patients was obtained specially for application in cerebrospinal fluid diagnostics.

Measuring Set-Up of FCS

A dual-color cross-correlation FCS set-up served as the basis for the aggregation measurements. The theoretical concept and practical set-up have been described in detail by Schwille in [23]. Based on this set-up, a prototype was developed on which the aggregation measurements were performed. For the SIFT measurements, the set-up was supplemented by a drive for the scanning of the sample and by a measuring card for intensity analysis.

The measuring set-up is schematically represented in FIG. 3. The beams of an Ar ion laser (488 nm) and of an He—Ne laser (633 nm) are coupled into the path of the rays in parallel through a single-mode glass fiber, an expanding lens and a double dichroic mirror and focused in the measuring solution by a microscope objective (×40 or ×63). The focuses of both beams constitute the open measuring volume of FCS. The aperture of the objective is completely illuminated, so that a radius of 0.25 μm (×40) or 0.19 μm (×63) results for the blue focus. The figure of the focuses is not completely ideal, since the radius of the red focus is larger than that of the green focus by about 20%, and the centers of the two focuses deviate by about 50 nm. However, the green focus is still completely within the red focus.

The fluorescent light is collected through the microscope objective and confocally imaged onto a pinhole. The pinhole can be controlled in terms of diameter and of x-y-z axes by step motors. The parallelized fluorescent light is split into red and green emissions by a dichroic mirror/filter combination and focused onto two avalanche photodiodes (APDs). The APDs have a detection efficiency of about 70% and produce a TTL pulse for each detected photon. Through an amplifier/diplexer, the TTL signal is passed on simultaneously to a hardware correlator card (ALV-5000, ALV, Langen, Germany) for correlation analysis and to a multichannel scaler-timer (MCS) card (MCD-2, FAST GmbH, Unterhaching, Germany, or C. Zeiss, Jena, Germany) for intensity analysis of the signal.

In the measurements, two objective/pinhole combinations were employed:
objective ×40/1.2 N.A. (Zeiss) and 50 μm pinhole;
objective ×63/1.2 N.A. (Olympus) and 30 μm pinhole.

Unless otherwise specified, the output powers of the excitation lasers were 57 μW (488 nm) and 53 μW (633 nm).

Scanning

For scanning the sample, the measuring solution was filled into a glass capillary of 50 mm length, 0.18 mm wall thickness and an interior cross-section of 2.6×0.2 mm. The sample volume was 20 μl. The ends of the measuring capillary were fixed on a glass slide by a colophony-based lacquer and simultaneously sealed.

The scanning of the measuring solution was effected by driving the positioning stage of the FCS measuring set-up (Märzhäuser, Wetzlar, Germany) through a macro language (WinBatch, Wilson Window Ware, Seattle Wash., USA). Within the Confocor controlling program (C. Zeiss, Jena), an array of 2×20 dots was defined whose spacing was 20 mm along the capillary direction and 10 μm in the capillary transverse direction. The dots of this array were accessed by moving the capillary on a meandering path relative to the microscope objective at a speed of 1 mm/s.

Intensity Analysis

Both the recording of the trace of fluorescence intensity and intensity analysis were effected on a separate measuring computer by an MCS card (C. Zeiss, Jena). Histograms of fluorescence intensity were established with the software Origin 6 (Microcal, Northampton, Mass., USA). A program for the automated establishing of intensity histograms was established and provided together with a measuring card by courtesy of Zeiss. The evaluation and graphic representation of the intensity histograms was performed by Perl routines.

Labeling of the Prion Protein

In order to minimize the influence of the fluorophor, conditions which led to an incomplete labeling of the protein were chosen in all labeling reactions, so that a maximum of one dye molecule was coupled to a protein molecule.

For labeling the PrP with the fluorescent dyes cyanine 5 (Cy5) as well as Oregon green 488 or Alexa488, an amino-reactive succinimidyl ester of the dye was coupled to a primary amino group of a lysine of the protein. An aliquot of the dye (about 100 μg) was dissolved in 50 μl of DMSO. 3 μl each of the dye was added to 100 μl of rPrP (90-231) (100 μg/ml) in NaP2 and stirred at RT in the dark for 1 h. Microspin columns (Mobitec) with Sephadex G-75 (Pharmacia) were equilibrated with 3*350 μl PBSS (centrifugation for 1 min, 750×g). After the reaction, the product was separated from excess dye over two microspin columns (centrifugation for 3 min, 750 g).

The proportion of labeled molecules was 4% for PrP-Oregon green and 14% for PrP-Cy5 when one fluorophor per protein molecule was assumed.

Labeling of the Antibodies

Microspin columns (Mobitec) with Sephadex G-15 (Pharmacia) were equilibrated with 3*350 of gel PBSN (centrifugation for 1 min, 750×g). 5-20 μl of antibodies (c=0.1-1 mg/ml) were filled with PBSN to 30 μl and transferred into PBSN buffer through the spin column (centrifugation for 3 min, 750×g). After the addition of 3 μl of NaC and 1.5 μl of Cy5 or 3 μl of Oregon green or Alexa488 (2 μg/μl in DMSO), the mixture was allowed to stand at 4° C. over night. The labeled antibodies were purified over a microspin column with Sephadex G-75 (Pharmacia) (3 min, 750×g) which had been equilibrated with PSBN. After renewed elution with 30 μl of PBSN, a second fraction of labeled antibodies was obtained. The concentration of the antibody and proportion of free dye (≦5%) were determined by auto-correlation measurement in FCS.

Determination of the Labeling Ratio

The protein concentration was determined by absorption measurement at 280 nm and a layer thickness of 1 cm in a spectrophotometer (Lambda 17, Perkin Elmer). From the absorption spectrum of the free dye, the absorption ratio α=E^{F}₂₈₀/EF^{F}₂₈₀ of the dye (Alexa488, Oregon green: E^{F}ₘₐₓ=495 nm; Cy5: E^{F}ₘₐₓ=650 nm) was determined for correcting the concentration of the labeled protein. The concentration of the labeled protein c_{P} is calculated according to:
cP=E28o(1−aEmax)′/(gll)11/m[M] (10)
where m is the molecular mass of the protein (g/mol). The concentration of the fluorophor C_{F} is calculated from the extinction coefficient of the dye, ε: c_{F}=E^{F}ₘₐₓ·ε(ε_{Oregon}=7.0·10⁴M⁻¹cm⁻¹, ε_{Alexa488}=7.1·10⁴M⁻¹cm⁻¹, ε_{Cy5}=2.5 ·10⁵M⁻¹cm⁻¹). The ratio of C_{F}/c_{P} represents the average number of fluorophors per protein molecule.
Determination of Concentration by FCS Measurement

The final concentration of the fluorescence-labeled probes and the proportion of free dye were determined by auto-correlation measurements. The structural parameter z₀/ω₀ and the diffusion time of the free dye were determined by auto-correlation measurements of Alexa488 and Cy5 dye solutions. The effective detection volume V≈1.3×4/3πω²₀z₀ was calculated from the measurement of a rhodamine green solution (D_{RG}=2.8·10⁻¹⁰m²s⁻¹) through ω₀=(4Dτ_{D})^{-1/2}. The size of the measuring volume was 0.4 fl (ω₀=0.25 μm, τ_{D}=55 μs) for the ×40 objective and 0.2 fl (ω₀=0.19 μm, τ_{D}=32 μs) for the ×63 objective.

SIFT Measurements

For the “scanning for intensely fluorescent target molecules” (SIFT), i.e., for the measurement on the diagnostic model system of CJD and AD, prion rods or Aβ aggregates in the stated concentration were diluted in cerebrospinal fluid or buffer in a silanized sample vessel (G. Kisker, Mühlhausen) to a volume of 18 μl. 2 μl of a mixture of fluorescence-labeled probes in PBSN was added, so that the final concentration of the probes was 6 nM (antibodies) or 10 nM (PrP). For measuring the cerebrospinal fluid samples from AD and CJD patients, 2 μl of probe mix was directly added to 18 μl of cerebrospinal fluid. A measuring capillary was filled with the sample without contamination, and subsequently sealed. The measurement was performed for 300 s or 600 s at 22° C. with a scanning speed of 1 mm/s. Contaminated material was decontaminated by autoclaving (2 h, 140° C.) or treatment with 2 M NaOH (minimum 2 h).

Aβ Aggregates

Aβ peptide (1-42) was supplied by Bachem Feinchemikalien (Heidelberg, Germany) in a lyophilized form. To produce preaggregated Aβ (1-42), the peptide was dissolved in DMSO (c=5 mg/ml), diluted in AS buffer to a concentration of 10 μM, and incubated at 22° C. for 2 h. Aliquots of the aggregation additive were diluted in PBSN to the stated concentration.

For adsorption measurement, aggregated Aβ (1-42) was first diluted in PBS to 10 μM. Aliquots were diluted 1:10 in the examined media, antibody mix was added (6E10-Cy5, pAB42-Alexa488, c=6 nM), filled into the measuring capillary and sealed. The measurement and storage were performed at 22° C. in a SIFT set-up (measuring time 300 s, bin width 500 μs, threshold 8·Iₘₐₓ).

Purification of Antibodies

Antibody 12F10 was purified by protein G affinity chromatography (MabTrap G II, Pharmacia) from serum-free cell culture supernatant. The column was rinsed with 5 ml of bidistilled H₂O and equilibrated with 3 ml of B buffer. 15 ml of culture supernatant to which 15 ml of B buffer had been added was charged onto the column through a membrane filter (0.45 μm, Millipore) using a sterile plastic syringe. The column was rinsed with 3 ml of B buffer until the absorption (E_{280 nm}) of the washing had decreased to the value of the buffer. Elution was performed with 4 ml of E buffer. The eluate was collected in 10 fractions in which 20 μl each of N buffer was charged in advance. The antibody was eluted in fraction 3 (400 μl). By absorption measurement, its concentration was determined to be 350 mg/ml. There were added 0.1% (v/v) of NP-40 and 0.005% of NaN₃, and the product was stored at −20° C.

Western Blot

For determining the concentration by Western blot, prion rods were diluted in cerebrospinal fluid from patients with no signs of neurodegenerative diseases. Scrapie-infected hamster brain (strain 263 K) was homogenized with 9 parts of lysis buffer and incubated with proteinase K (100 μg/ml) for 30 min at 37° C. The digestion was stopped by the addition of 5 mM PMSF and boiling in charging buffer. 10 μl was separated on a 12.5% SDS polyacrylamide electrophoretic gel. After transfer to a nitrocellulose membrane (0.45 μm, Bio-Rad, CA), the PrP was detected by incubation with 3F4 as a primary antibody and alkaline phosphatase coupled goat-anti-mouse secondary antibody. The phosphatase activity was visualized by the CDP-Star chemiluminescence system (Tropix Inc., Bedford, Mass.) on Hyperfilm ECL (Amersham, Ill.) according to the manufacturers' directions. The detection of rPrP was effected analogously, but without PK digestion. If required, the PA gel was subsequently stained with Coomassie blue (30 min, RT).

Development of a Cerebrospinal Fluid Diagnostic Method for CJD and Alzheimer's Disease

Aggregation with conversion of the secondary structure into a more hydrophobic conformation is a basic characteristic of the prion protein. As with Alzheimer's disease, which leads to the formation of pathological aggregates of the Aβ peptides, the detection of aggregated protein can form the basis of a diagnostic test. For this purpose, it is desirable to detect individual pathological aggregates.

Attachment to Aggregation Nuclei

By adding monomeric fluorescence-labeled PrP to a solution of multimeric aggregates, the attachment process of the monomers can be made visible. Also in the course of de-novo aggregation, fluorescence peaks which could be assigned to individual multimeric aggregates of the prion protein with a large number of bound dyes increasingly appeared. The passing of such aggregates through the focal volume produces a shower of fluorescence photons, briefly referred to as a burst in the following, by which the aggregates can be immediately detected (see FIG. 4).

However, while self-aggregation of the prior protein in a concentration range which is relevant to FCS resulted in a detectable quantity of multimers only within a period of ≧30 min, the attachment to preexisting aggregates was quantitative already within the sample preparation time, i.e., within a few minutes. In the further course of measurement, the number of detected aggregates per unit time remained constant (bottom of FIG. 4).

On the basis of these results, the following strategies for the labeling of aggregated target molecules suggested themselves:

1. Co-aggregation of homologous fluorescent monomers (PrP or Aβ)

2. Co-aggregation of heterologous fluorescence-labeled monomers

3. Binding of specific fluorescence-labeled antibodies

4. mixed approach with monomers and antibody probes

The labeling can be effected with either one or two different probe molecules labeled with different fluorescent dyes. The labeling strategy determines the analytical method by which the signal of the fluorescence-labeled aggregates can be detected and quantified. The development of a diagnostic system for prion diseases and Alzheimer's disease can proceed from the same basic idea, the attachment of probes to an aggregation nucleus. Its development is shown in the following.

Separation of the Signal from the Aggregates

By a classical correlation analysis of the fluctuation of the fluorescence signal, the diffusion movement of individual molecules can be evaluated quantitatively. This involves the determination of the average fluctuation time from a large number of molecular passages. When only a few passages of highly labeled aggregates are detected during an individual measurement, the measured passage time depends not only on the aggregate size, but also critically on the path traveled by the individual particles through the measuring volume. Therefore, the aggregate size can only be estimated from the passage time. At a probe concentration of 10 nM, the free probes are in a 10³fold to 10⁶fold excess over the aggregates. FIG. 5 shows the passage of a single aggregate of recombinant prion protein which was detected with one probe in auto-correlation FCS or with two differently labeled probes in cross-correlation FCS. The proportion of the aggregate was <10% of the auto-correlation signal. By heterologous detection using a combination of green-labeled rPrP as one probe and an aggregate-specific PrP antibody (15B3) as another probe, the signal from multimeric aggregates could be completely separated from the signal of monomeric and oligomeric PrP molecules (see FIG. 5, left). Passage times of the aggregates were determined to be from 3 to 50 ms. The average diffusion time corresponds to a molecular weight of several MDa.

The intensity of the labeled target molecules is an average of 20 to 50 times the intensity of the free probe molecules. Thus, this corresponds to the minimum number of probe molecules bound to one aggregate. Since the course of the fluorescence intensity of aggregation allows to conclude on the monomers' being quenched in a bound state, the actual number of bound probes is probably higher at least when monomers are used. Due to the large number of bound fluorophors, single molecular passages can be detected immediately.

However, for a concentration of the aggregates in the subpicomolar range, only a few target molecules can be detected in a sample in this way. For a 1000 mer at a femtomolar concentration and with a focus diameter of 0.4 μm, a frequency of entry of 0.5·10⁻³s⁻¹ results, which corresponds to about two particles per hour [3]. Thus, the number of passages of aggregates through the measuring focus becomes the limiting factor, which is again limited by the slow diffusion of the aggregates.

Thus, the fluorescence intensity and cross-correlation are two available parameters with which individual target molecules can be detected even for a high excess of free probes.

In experiments for detecting pathological aggregates of the prion protein in the cerebrospinal fluid from Creutzfeldt-Jakob patients, the number of labeled aggregates was first determined directly from the number of signal peaks in the intensity trace of the fluorescence signal. As a probe, solubilized prion protein derived from the brain material of scrapie-infected hamsters was used. The PrP probe was labeled with the fluorophor Cy2. FIG. 4 shows a section from the trace of the fluorescence signal from a measurement of the cerebrospinal fluid from a Creutzfeldt-Jakob patient in a single-channel FCS set-up.

The fluorescence signal was recorded by the software of the FCS appliance and in parallel by a multichannel scaler (MCS) card. Several fluorescence peaks which indicate the passage of a highly labeled macromolecule through the measuring focus can be seen. For the detection of dementia-specific aggregates of Aβ peptide in the cerebrospinal fluid from Alzheimer's patients, a successful application of this method has been described [16]. In the present system, the low number of events and probe-inherent aggregates did not allow a reproducible distinction between the cerebrospinal fluid samples from CJD patients and those from control patients suffering from different neurodegenerative diseases.

Quantitative Intensity Analysis

The direct counting of peaks in the fluorescence signal without quantification of a threshold value of intensity only allows for a relatively unreliable identification of labeled target molecules. Therefore, a simple form of intensity analysis was developed which represents the proportion of fluorescence signal having a high intensity in an intensity histogram in order to quantitatively determine the proportion of the peak signal thereby. For this purpose, the signal from the photodetector is split, and the fluorescence photons are summed up in intervals of equal length (bins) in a counter-timer card in parallel with correlation analysis. The number of time intervals with a particular number of detected fluorescence photons is represented on-line in an intensity histogram in the course of measurement.

The intensity distribution of the free probe molecules (FIG. 6d) is well defined by the homogeneous average diffusion time of the probe molecules and the number of fluorophors. When target molecules having bound thereto a large number of fluorescent probes were also present in the solution in addition to free probes, then the intensity histogram showed a proportion of measuring channels having a high number of detected fluorescence photons (FIG. 6b).

The distribution of fluorescence intensity is produced by the convolution of fluctuation of the number of molecules with the excitation and detection characteristics of the measuring set-up, the so-called collection efficiency function (CEF) [19]. Experimentally, the intensity distribution of the antibodies (3F4-Alexa488) could be well fitted by a log normal distribution (FIG. 7a).

The component of the labeled aggregates was less well-defined due to the heterogeneous aggregate size. As shown by FIG. 7b), it could be described but imperfectly by a single distribution term. It could be quantified by a superposition of the distributions for different aggregate sizes and numbers of chromophores. However, due to the small N of the detected aggregates, this appeared hardly practicable. Therefore, the signal from the target molecules was separated from the signal of the probes by setting as threshold value and quantified. In this method, part of the signal from the target molecules is lost because it overlaps with the distribution of the probe molecules. The higher the threshold value, the higher of course is the proportion of target molecules whose signal remains below the threshold and therefore are not detected. As a rule, a threshold of 3σ is chosen for the separation of background noise [1]. Since the probe molecules were present in an excess of up to 106 as compared to the target in the present case and a false positive assignment of the signals was nevertheless to be avoided, a substantially more conservative threshold value was selected, which was typically around 12σ for detection and 8σ for the characterization of aggregates.

The separation of the signal from probe and target molecules in the intensity histogram depends on time resolution, i.e., the bin width. For a maximum separation from the probe background, the entire photons from the passage of one target molecule should fall into one bin. Thus, this is the minimum time resolution of detection. When the bin width is larger than the average dwelling time, the signal-to-noise ratio decreases by averaging across the probe background. When the molecular passage is distributed onto too many bins, the relative fluctuation of the probe signal increases and thus reduces the signal-to-noise ratio. In the case of diffusion-controlled movement, the passage time is about four times the average diffusion time τ_{diff}. In the case of a straight flow, it is determined by the ratio of focal diameter and flow rate. Therefore, for the measurement with a moved sample, a bin width of 0.5 ms was chosen for a traveling speed v=1 mm/s and a focal radius ω₀=0.5 μm, so that the signal of a target molecule is distributed onto 1-2 bins.

Sensitivity Enhancement by Moving the Measuring Volume

While allowing for a simple separation and quantification of the signal from the target molecules, intensity analysis does not increase the number of molecular passages and thus the sensitivity of detection. However, like cross-correlation analysis, it yields a parameter for the direct distinction between bound and unbound probe molecules, so that the size information which is yielded by the diffusion time is no longer required for recognizing the target molecules. These can be recognized even when the sample is moved relative to the measuring focus during measurement.

By “scanning” the sample, the diffusion movement of the molecules was superposed by a “flow movement”. For molecules whose diffusion-caused frequency of entry into the volume element is small as compared to its dwelling time in the measuring volume, the detection sensitivity can be critically increased by increasing the measuring volume, i.e., by “scanning” the sample.

In contrast to stationary measurement, in which the measuring solution usually rested as a drop on a cover slide, the sample solution, for measurement with a moved volume element, was filled into a drawn glass capillary which enclosed a volume of 20 μl. The sealed measuring capillary was moved in a meandering way during measurement at a speed of 1 mm/s, and the sample volume was thus covered. The passing time of the aggregates through the measuring volume was reduced from 3-50 ms to about 0.5 ms by the “scanning” of the sample. Therefore, the passing time was solely determined by the flow rate and thus by the geometry of the measuring volume. Thus, the number of measuring channels with a high intensity signal also became proportional to the number of labeled particles passing through the measuring volume.

Due to the low number of events in stationary measurement, the increase in sensitivity by the sample movement could not be measured directly with the diagnostic system because few or no aggregates were usually detected in the stationary sample. To determine the enhancement of the passing frequency by the “scanning” of the sample, fluorescent polystyrene beads having a diameter of 0.1 μm served as a model of the aggregates. The average diffusion time of the beads, which was about 3 ms, corresponded to the lower limit of diffusion times which had been determined for the PrP aggregates. In the moved measurement (FIG. 8), about 100fold more events were detected as compared to stationary measurement.

The number of detected events increases with the speed with which the sample is moved. If the diffusion-caused movement is neglected, the number of detected events is proportional to the covered volume. When pathogenic PrP^{Sc} aggregates were detected, an increase in scanning speed from 1 mm/s to 5 mm/s increased the number of events and thus sensitivity by a factor of three. However, in routine use, the type of drive for the positioning stage limited the movement to 1 mm/s.

Evaluation of the SIFT Method with PrP and Antibody Probes

The combination of intensity analysis with a sample movement, i.e., the scanning for intensely fluorescent targets (SIFT), was examined on a model system with respect to detection sensitivity. Purified aggregates of the pathogenic prion protein obtained from the brain tissue of Syrian hamsters, so-called prion rods, were diluted in cerebrospinal fluid. For detection, on the one hand, fluorescence-labeled recombinant hamster PrP, and on the other hand, a labeled PrP-specific monoclonal antibody were used. The fluorescence signal was evaluated in an intensity histogram (see FIG. 9A, C), and the numbers of channels having an intensity above a threshold of 500 photons/channel were added (see FIG. 9B, D). Depending on the concentration of the prion rods, a high intensity signal was obtained. However, already without the addition of target molecules, the PrP probe showed a proportion of high intensity signal before which as a background the prion rods could be detected only down to a dilution of about 1:500. The probe-inherent signal, which could not be observed in stationary measurements due to their low sensitivity, is caused by aggregates of the PrP probe molecules. These can be formed by two processes: on the one hand, by the self-aggregation of PrP, and on the other hand, by the formation of high molecular weight aggregates during the labeling of PrP with the fluorescent dye. The aggregate formation during the labeling could not be completely suppressed despite of carefully choosing the reaction conditions. Multimeric PrP aggregates can be detected in the moved sample with considerably higher sensitivity as compared in the measurements for self-aggregation. In contrast, the antibody probe was substantially free from inherent signal, so that the detection threshold could be decreased by two orders of magnitude by using a monoclonal antibody.

FIG. 10 shows the SIFT signal as a function of the prion rod concentration for different threshold values. While the number of channels with high intensity changed depending on the threshold value, the choice of the threshold value had no influence on the proportionality of the SIFT signal to the concentration of prion rods.

Two-Dimensional Intensity Analysis

To increase the specificity of detection, the detection system was extended by a second probe directed against a different epitope of the prion protein. It was labeled with a second fluorescent dye which can be excited in the red spectral region at 633 nm. Binding of the probes yields target molecules which bear a high number of both dyes. Thus, two parameters can be utilized for isolating the signal from the target molecules:

1. the amplitude of dual-color cross-correlation; and

2. the simultaneous fluorescence intensity.

If the fluorescence signal is observed with a high time resolution, the passage of a doubly labeled aggregate can be identified by a peak in the fluorescence signal which occurs simultaneously in both measuring channels. For an intensity analysis of the two detection channels, the fluorescence signal of the two channels was plotted in a two-dimensional intensity histogram. By analogy with the intensity analysis of one measuring channel, the fluorescence photons were counted in parallel in two channels in bins of 500 μs, and the intervals were summed up in a two-dimensional array in accordance with the number of detected photons. In an intensity histogram which can be represented on-line during the measurement, the fluorescence intensity of the two colors is plotted on the axes, and the number of bins of an intensity pair is represented in a logarithmic fashion by the color of the respective dot. FIG. 11 shows the intensity histogram of a measurement of prion rods, superposed by a schematic representation of the signal ranges.

By this evaluation, the signal of particles which simultaneously produce high intensity signals in both the green and the red detection channel is separated from the signal of the free probe. The aggregate-specific signal lies in the fourth quadrant of the histogram, while the majority of the bins represents the combined signal distribution of the two free probes and thus lies in the first quadrant (see FIG. 11, with gray outline). Again, the high intensity signal is separated through a threshold value in the simplest case. In order to account for the crosstalk in the detection channels, a progressive threshold value was selected. If a high intensity signal is detected in a bin in one of the channels, the threshold value for separating the specific signal will increase in the other channel (see FIG. 11, green lines). Strictly speaking, this is required only for delimiting the specific signal from a high intensity green non-specific signal. The emission spectrum of the green dye overlaps to a low extent with the emission spectrum of the red fluorophor. Therefore, about 0.5% of the photons which a molecule bearing only green fluorophors emits when passing through the focus are detected by the red detector.

By the simultaneous labeling with two types of probe molecules, the specificity of detection could be increased. Both probes, which were directed against different epitopes of the target molecule, independently bound to the aggregate. At the same time, in part, there was non-specific binding of the probes to cellular components in the sample solution and binding by secondary proteins, e.g., secondary antibodies, present in the biological sample. These processes resulted in the formation of intensely fluorescent particles. In the measurement represented in FIG. 11, this was the case for the red-labeled antibody probe. However, whether such non-specific aggregates occurred in one or two channels depended on the sample examined and on other factors which are difficult to control, such as the antibody preparation. This signal which occurs in only one of the measuring channels (see FIG. 11, red and green ellipses) can also be distinguished from the specific signal in dual-color intensity analysis.

Evaluation of Specificity and Sensitivity

The extended detection system was again evaluated on a diagnostic model system with respect to specificity and sensitivity of detection. For this purpose, cerebrospinal fluid from control patients to which prion rods had been added was used.

The specificity of recognition of the target molecule was examined by specific and non-specific probes and specific and non-specific target molecules (see FIG. 12). As specific probes, two monoclonal antibodies were respectively employed. Without the addition of prion rods, almost no simultaneous high intensity signal was observed (see FIG. 14). Also after the addition of aggregated Aβ(1-42) peptide as a non-specific target molecule, no doubly labeled aggregates were detected, even though one antibody (3F4) showed a non-specific signal (FIG. 12b). In contrast, when specific probes were added, 3F4 and 12F10 to PrP target molecules and 6E10 and pAB42 to Aβ(1-42) peptide, a strong signal of doubly labeled target molecules occurred (FIG. 12a, c). In order to check whether two non-specific probes would simultaneously bind to a single target molecule, two probes against the target molecules 1L8 and Aβ(1-40), which are not related to Creutzfeldt-Jakob disease, were added to control cerebrospinal fluid containing prion rods. Although a high intensity signal proportion, which may have been formed by binding or aggregation of the probes, was observed in both channels, no high intensity signal was observed to occur simultaneously in both channels (FIG. 12d).

The sensitivity of the detection system was compared with the detection of prion protein by Western blot upon digestion with proteinase K. Virtually all current tests for pathogenic prion protein are based on this method. Aliquots of the prion rod material diluted in cerebrospinal fluid were analyzed in parallel by Western blot and measured in a confocal fluorescence-spectroscopic set-up, the signal being evaluated by SIFT and cross-correlation analysis (see FIG. 13). FIG. 14 shows the intensity histograms for different concentrations. The concentration of the prion rods could be measured by two-channel intensity analysis through four orders of magnitude, the detection threshold being at a dilution of 1:2·10⁵. In contrast, the detection threshold of the Western blot was at 1:10⁴. The signal of the Western blot was quantified by gel densitometry from the band intensity. By comparison with the brain tissue of a scrapie-infected hamster applied in parallel, it was established that the detection limit of the blot corresponded to about 1 μg of brain tissue. This amount of tissue contains about 10 pg of monomeric PrP^{Sc}[17], which corresponds to a concentration of 33 pM for the applied quantity of 20 μl. Accordingly, the detection limit of the SIFT measurement, in which from one to two aggregates were still detected in a measuring time of 600 s, was 0.5 pg or 2 pM PrP^{Sc}.

The physical detection threshold of the measurement is the detection of a single particle in the covered volume. For a scanning volume of about 2·10⁶ focal volumes of the confocal set-up, this corresponds to a concentration of 1 fM when distortions of the volume element are neglected. The aggregate concentration, which results from the SIFT measurement from considerations relating to the detection threshold, can be related to the concentration of monomeric PrP which was determined in the Western blot. This results in an average aggregate size of about 1000 PrP molecules.

The sensitivity in the detection of pathologic amyloid aggregates from Alzheimer's disease, whose main components are Aβ peptides having a length of 40-43 amino acids, was examined analogously on Aβ(1-42) peptide, which had previously been aggregated under controlled conditions. Two antibodies of which one specifically recognized the C-terminal amino acids of Aβ42 while the other recognized an epitope in the consensus sequence of Aβ peptides served as fluorescent probes. The Aβ aggregates could be detected down to a concentration of 100 pM (9 pg) of monomeric Aβ42. This allows to conclude on an aggregate size of about 10⁵ units per aggregate.

Comparison with Cross-Correlation Analysis

In parallel with the evaluation using SIFT, the fluorescence signal of the measurements was evaluated by cross-correlation of the detection channels according to equation 2. FIG. 15 shows the cross-correlation curves of a dilution of prion rods in cerebrospinal fluid. Within a range of from 0 to 50 ng of PrP (160 pM), the cross-correlation amplitude is proportional to the amount of prion rods employed. Due to the high number of fluorophors bound to one target molecule, the effective detection quantum yield (cpms), whose measure is the count of detected photons per molecule and per second, is up to 200 kHz and thus a multiple of the value of 1-2 kHz achieved by doubly labeled oligomers in the experiments relating to self-aggregation in the cross-correlation signal. The high molecular detection efficiency causes a high signal-to-noise ratio and thus a high cross-correlation amplitude. The cross-correlation amplitude could be differentiated from the signal of the control sample down to a PrP concentration of 5 pM. Due to the low number of events, the correlation amplitude was highly scattered for low concentrations.

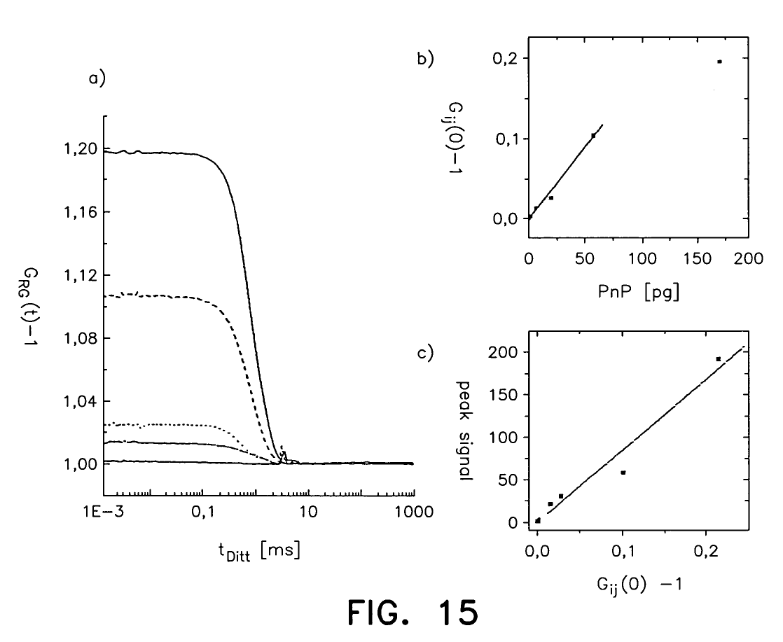

Within one measuring series, both parameters, Gᵢⱼ(0) and SIFT signal, were proportional (see FIG. 15c). For the two methods to serve as a general measure of concentration, it is additionally required that the two signals correlate independently of the measuring conditions, such as buffer and detergent concentrations. FIG. 16 compares the ratio of the two signals under a variety of buffer conditions for the model system of the aggregated Aβ peptide. It remained constant for a pair of probes when the excitation was constant.

Application in the Diagnostics of Cerebrospinal Fluid

In particular, the invention discloses a diagnostic system for the highly sensitive detection of pathological aggregates for the diagnosis of Creutzfeldt-Jakob and Alzheimer's diseases. Cerebrospinal fluid suggests itself as the medium to be examined for three reasons: First, the cerebrospinal fluid bathes the central nervous system of humans. Thus, unlike blood, it is not separated from the site of production of the pathologic aggregates by the blood-brain barrier. Second, cerebrospinal fluid is a “clean” medium. It hardly contains any cells or proteins which absorb in the range of excitation wavelengths of are fluorescent themselves, and it is thus well suitable for fluorescence-spectroscopic measurements. Third, it can be obtained relatively simply and without a risk from patients by a spinal puncture. For detecting neurodegenerative secondary markers, such as the 14-3-3 protein, this is done within the scope of clinical routine examinations.

Detection of Pathogenic PrP in the Cerebrospinal Fluid of CJD Patients

The detection approach, which was developed and evaluated on the model system of the prion rods, also served for detecting pathological aggregates of the prion protein in the cerebrospinal fluid of Creutzfeldt-Jakob patients. The cerebrospinal fluid samples were directly mixed with the probe mix and measured for 600 s in the two-channel SIFT measuring set-up. In five out of 24 cerebrospinal fluid samples from the patients whose CD diagnosis was ascertained due to clinical or neuropathological criteria, a specific signal could thus be detected which corresponded to the simultaneous binding of the two probes to one PrP^{Sc} aggregate. A collective of patients suffering from other neurodegenerative diseases served as a control group in order to ensure that the test was specific for CJD and not just recognized some secondary effect of neurodegenerative diseases. From none of the samples of the control patients, a signal was obtained which was specific for PrP^{Sc}(see FIG. 17). This formally corresponds to a sensitivity of 21% and a specificity of 100% for the detection of Creutzfeldt-Jakob disease. This is as yet the highest value for a pathogen-specific test in the cerebrospinal fluid of a patient [2].

Cerebrospinal Fluid Diagnostics of Alzheimer's Disease

Alzheimer's disease is characterized by an increased formation of fragments of a transmembrane protein, the so-called amyloid precursor protein (APP), which aggregate in a consequent process and form amyloid depositions. Unlike the pathological PrP^{Sc}, the amyloid Aβ peptides can also be detected in low quantities in healthy humans as normal metabolites. Therefore, a pathologically increased amount of aggregated peptides is to be defined by a threshold value.

Using two-channel intensity analysis, untreated cerebrospinal fluid samples from six Alzheimer's patients and 16 samples from patients suffering from other neurodegenerative diseases and from healthy patients were examined. The antibody probe system was used which had been established and evaluated on the basis of artificial Aβ42 aggregates. In 83% (5 out of six cases) of the cerebrospinal fluid samples from Alzheimer's patients examined, the amount of aggregate-specific signal was above the set threshold value. In contrast, the signal from all control patients was lower (see FIG. 18).

In the case of both Alzheimer's and Creutzfeldt-Jakob patients, the cerebrospinal fluid samples examined were those obtained within the scope of clinical routine examinations, such as the detection of the neurodegenerative secondary marker 14-3-3. Therefore, they are very heterogeneous with respect to their clinical history. A series of five AD samples and 4 control cerebrospinal fluid samples, which had been expressly put in safekeeping, were examined. Here, a significantly higher amount of amyloid aggregates could be detected in the AD-positive samples as compared to the samples from clinical routine diagnostics, which stresses the significance of sample preparation.

Differentiation of Prion Strains

For the detection of pathological aggregates within the scope of diagnostic systems, the sole result evaluated in the two-channel intensity analysis was whether an aggregate was labeled with a high number of both probe molecules. In contrast to correlation analysis, which yields information about the average concentration and the degree of labeling of the detected molecules, intensity analysis covers the signal of each detected particle separately. Therefore, it would allow to determine the ratio of the signal in the two measuring channels and thus the ratio of bound probes for each target molecule. Thus, in addition to the detection of aggregates, their characterization from the relative affinity of several probes was also possible.

The differential binding of a number of different monoclonal antibody probes to pathological prion protein was examined on purified human PrP^{Sc}. It was possible to differentiate different types of pathological prion protein. The measurement was effected in the same measuring set-up as the diagnostic application. To determine the ratio of the signal from the two fluorescence-labeled probes in one measurement, the intensity histogram of the two-channel intensity analysis was divided into sectors having the same signal ratio. In each sector, the number of measuring channels whose intensity was above the threshold value was determined (FIG. 20). The aggregate-specific signal was separated through a threshold of 8·σ. The high intensity signal could be securely separated from the probe signal using this threshold. In contrast, a noise signal from the non-specific probe aggregation was not separated from the residual high intensity signal. For this reason, the sector in which the signal with a red probe proportion of 0-10% is summarized contains a proportion of non-specific signal in all measurements. This proportion was determined in reference measurements and subtracted from the aggregate-specific signal.

The separation of prion types was optimized with various probe pairs and detergent additives. Purified pathogenic prion protein of type 1 and type 11 of two patients homozygotic at codon 129 M7M could be characterized by the relative probe binding. Both conformations could be reproducibly differentiated by the binding ratio of probes (FIG. 21a). The aggregate-specific signal of type 11 (129 M/M) shows a normal distribution around a probe ratio of 45±15%. The additional signal with a green proportion of ≧90% can probably be attributed to aggregation or cross-linking of the probe. In contrast, the labeling ratio in PrP^{Sc} type I is shifted in favor of the green-labeled probe (mAB 917Alexa). After subtraction of the probe-inherent signal proportion, the distribution maximum is at a proportion of green-labeled probe of 85±20%.

To ensure that the differentiation was made due to the conformation of the target molecule rather than secondary effects, such as contaminations from sample processing, a mixture of type I and type II of PrP^{Sc} was analyzed. Although the distributions of the two types superpose, the overall distribution of the mixture is essentially congruent with the sum of intensity distributions obtained from individual measurements (FIG. 21b). Thus, the different affinities of the probes can be attributed to the conformation of the target molecule rather than to contamination effects. When the distribution maximum of the prion types has been determined by gauging measurements, the proportions of type I and type II PrP^{Sc} in the samples can be determined without complete separation.

To differentiate the signal from the PrP^{Sc} types, it is not required to detect each particle with the same efficiency. In contrast to other methods for the characterization of individual particles by the relative binding affinity of several probes, such as FACS analysis on the cellular level, the quantitative detection of the fluorescence signal on the basis of single molecular passages yields an internal standard for the determination of the labeling ratio.

### LIST OF ABBREVIATIONS

| | |
|---|---|
| Ac | acetate |
| AD | Alzheimer's disease |
| Alexa488 | Alexa Fluor ™ 488 (commercial name of a rhodamine |
| | derivative) |
| APP | amyloid precursor protein |
| AS | amino acid |
| BSA | bovine serum albumin |
| BSE | bovine spongiform encephalopathy |
| CD | circular dichroism |
| CJD | Creutzfeldt-Jakob disease |
| Cy2/Cy5 | FluoroLink ™ cyanine 2/cyanine 5 |
| Da | Dalton |
| DNA | deoxyribonucleic acid |
| DMSO | dimethyl sulfoxide |
| ε | extinction coefficient |
| FCS | fluorescence correlation spectroscopy |
| FITC | fluorescein isothiocyanate |
| g | acceleration due to gravity |
| Gl. | equation |
| GPI | glycosylphosphaditylinositol |
| h | hour(s) |
| IgG | immunoglobulin |
| M | molar |
| min | minute(s) |
| NMR | nuclear spin resonance |
| NP-40 | non-ionic alkylphenylpolyoxyethylene detergent |
| | (commercial name) |
| nvCJD | new variant of Creutzfeldt-Jakob disease |
| PAGE | polyacrylamide gel electrophoresis |
| PK | proteinase K |
| PMSF | phenylmethylsulfonyl fluoride |
| PrP | prion protein |
| PrP^{c} | cellular prion protein |
| PrP^{Sc} | pathologic scrapie isoform of prion protein |
| RNA | ribonucleic acid |
| rpm | revolutions per minute |
| rPrP | recombinant prion protein |
| RT | room temperature |
| SDS | sodium dodecylsulfate |
| Tab. | table |
| TSE | transmissible spongiform encephalopathy |
| Tris | tris(methylamino)methane |
| IN. | over night |
| UV | ultraviolet |
| w/v | parts weight per volume |
| ZNS | central nervous system |

### REFERENCES

[1] Barnes M. D., Whitten W. B., Ramsey J. M. (1995), Detecting Single Molecules in Liquids, Analyt. Chem. 367, 418A-423A.
[2] Brown P, Gibbs C. J., Rodgers-Johnson P, Asher D. M., Sulima M. P., Bacote A., Goldfarb L. G., Gajdusek D. C. (1994), Human spongiform encephalopathy: the National Institute of Health series of 300 cases of experimentally transmitted disease, Ann. Neurol. 35, 513-529.
[3] Eigen M., Rigler R. (1994), Sorting single molecules: application to diagnostics and evolutionary biotechnology, Proc. Natl. Acad. Sci. USA 91, 5740-5747.
[4] Eigen M. (1996), Prionic or the kinetic basis of prion diseases, Biophys. Chem. 63, A1-18.
[5] Kask P., Kaupo P., Ullmann D., Gall K. (1999), Fluorescence-intensity distribution analysis and its application in biomolecular detection technology, Proc. Natl. Acad. Sci. USA 13756-13761.
[6] Kettling U., Koltermann A., Schwille P., Eigen M. (1998), Real-time enzyme kinetics monitored by dual-color fluorescence cross-correlation spectroscopy, Proc. Natl. Acad. Sci. USA 95, 1416-1420.
[7] Kasesak R. J., Rubenstein R., Merz P. A., Tonna-DeMasi M., Fresko R., Carp R. I., Wisniewski H. M., Diringer H. (1987), Mouse polyclonal and monoclonal antibody to scrapie-associated fibril proteins, J. Virol. 61, 3688-3693.
[8] Krasemann S., Groschup M., Harmeyer S., Hunsmann G., Bodemer W. (1996), Generation of monoclonal antibodies against human prion protein in PrP^{0/0} mice, Mol. Med. 2, 725-734.
[9] Magde D., Elson E. L., Webb W. W. (1972), Thermodynamic fluctuations in a reacting system, measurement by fluorescence correlation spectroscopy, Phys. Rev. Lett. 29, 705-711.
[10] Magde D., Elson E. L., Webb W. W. (1974), Fluorescence correlation spectroscopy II. An experimental realization, Biopolymers 13, 29-61.
[11] Magde D., Webb W. W., Elson E. L. (1978), Fluorescence correlation spectroscopy III. Uniform translation and laminar flow, Biopolymers 17, 361-376.
[12] Maiti S., Haupts U., Webb W. W. (1997), Fluorescence correlation spectroscopy: Diagnostics for sparse molecules, Proc. Natl. Acad. Sci. USA 94, 11753-11757.
[13] Mehlhorn I., Groth D., Stockel J., Moffat B., Reilly D., Yansura D., Willett W. S., Baldwin M., Fletterick R., Cohen F. E., Vandlen R., Henner D., Prusiner S. B. (1996), High-level expression and characterization of a purified 142-residue polypeptide of the prion protein, Biochemistry 35, 5528.
[14] Müller W. E. G., Ushijima H., Schröder H. C., Forrest, J. M., Schatton W. F., Rytik P. G., Heffner-Lauc M. (1993), Cytoprotective effect of NMDA receptor antagonists an prion protein (PrP^{Sc})-induced toxicity in rat cortical cells cultures, Eur. J. Pharmacol. 246, 261-267.
[15] Oehlenschläger F., Schwille P., Eigen M. (1996), Detection of HIV-1 RNA by nucleic acid sequence-based amplification combined with fluorescence correlation spectroscopy, Proc. Natl. Acad. Sci. USA 93, 12811-12816.
[16] Pitschke M., Prior R., Haupt M., Riesner D. (1998), Detection of single amyloid B protein aggregates in the cerebrospinal fluid of Alzheimer's patients by fluorescence correlation spectroscopy, Nature Medicine 4, 832-834.
[17] Prusiner S. B., McKinley M. P., Bowman K. A., Bolton D. C., Bendheim P. E., Groth D. F., Glenner G. G. (1983), Scrapie prions aggregate to form amyloid-like birefringent rods, Cell 35, 349-358.
[18] Qian H., Elson E. L. (1990), Distribution of molecular aggregation by analysis of fluctuation moments Proc. Natl. Acad. Sci. USA 87, 5479-5483.
[19] Qian H. (1990), On the statistics of fluorescence correlation spectroscopy, Biophys. Chem. 38, 49-57.
[20] Riesner D., Kellings K., Wiese U., Wulfert M., Mirenda C., Prusiner S. B. (1993), Prions and nucleic acids: search for “residual” nucleic acids and screening for mutations in the PrP-gene, Dev. Biol. Stand. 80, 173-181.
[21] Rigler R., Mets Ü., Widengren J., Kask P (1993), Fluorescence correlation spectroscopy with high count rate and low background: analysis of translational diffusion, Eur. Biophys. J 22, 169-175.
[22] Safar J., Wille H., Itri V, Groth D., Serban H., Torchia M., Cohen R E., Prusiner S. B. (1998), Eight prion strains have PrP3″ molecules with different conformations, Nature Medicine 4, 1157-1165.
[23] Schwille P. (1997), Fluoreszenz-Korrelations-Spektroskopie: Analyse biochemischer Systeme auf Einzelmolekulebene, Dissertation, Braunschweig
[24] Schwille P. Meyer-Almes F.-J., Rigler R. (1997), Dual-color fluorescence cross-correlation spectroscopy for multicomponent diffusional analysis in solution, Biophys. J. 72, 1878-1886.
[25] Schwille P., Bieschke J., Oehlenschläger F. (1997), Kinetic investigations by fluorescence correlation spectroscopy: The analytical and diagnostic potential of diffusion studies, Biophys. Chem. 66, 211-228.
[26] Schwille P., Haupts U., Maiti S., Webb W. W. (1999), Molecular dynamics in living cells observed by fluorescence correlation spectroscopy with one- and two-photon excitation, Biophys. J. 77, 2251-2265.
[27] Thompson N. L. (1991), Fluorescence correlation spectroscopy, in: Topics in fluorescence spectroscopy, ed. Lakowicz J. R., Vol. 1, Plenum Press, New York, S. 337.
[28] World Health Organisation, Human spongiform encephalopathies, Weekly epidemiological record 1998 73; 47 361-265.
[29] Winkler T., Kettling U., Koltermann A., Eigen M. (1999), Confocal fluorescent coincidence analysis: An approach to high throughput screening, Proc. Natl. Acad. Sci. USA 96, 1375-1378.
[30] Zerr I., Bodemer M., Racker S., Grosche S., Poser S., Kretzschmar H. A., Weber T. (1995), Cerebrospinal fluid concentration of neuron-specific enolase in diagnosis of Creutzfeldt-Jakob disease., Lancet 345, 1609-1610.
[31] Zerr I., Bodemer M., Gefeller O., Otto M., Poser S., Wiltfang J., Windl O. Kretzschmar H. A., Weber T. (1998), Detection of 14-3-3 Protein m the cerebrospinal fluid supports the diagnosis of Creutzfeldt-Jakob disease, Ann. Neurol. 43, 32-40.

1-19. (canceled)

20: A method for the determination and individual characterization of particles by means of at least two different fluorescence-labeled probes in a sample, characterized in that
the particles have at least one binding site for at least one of the at least two different fluorescence-labeled probes,
the at least two different fluorescence-labeled probes are present is the sample,
the number of the fluorescence-labeled probes bound to the particles is measured by a measuring method based on an implemented set-up for dual-color fluorescence spectroscopy which scans for intensively fluorescence particles,
determination of particles from the mutual ratio of quantities of probes bound to the particles.

21: The method according to claim 20, characterized in that the particles are molecules or molecular aggregates.

22: The method according to claim 20, characterized in that the mutual ratio is measured in a measuring volume which is a subvolume of the sample.

23: The method according to claim 22, characterized in that the measuring volume is ≦10⁻¹²1.

24: The method according to claim 22, characterized in that the measuring volume is ≦10⁻¹⁴1.

25: The method according to claim 20, characterized in that the measuring method based on an implemented set-up for dual-color fluorescence spectroscopy is effected by producing a constant relative movement between the sample and the measuring volume.

26: The method according to claim 20, characterized in that the relative movement is realized by a lens system which allows movement of the measuring volume, by movement of a sample carrier holding the sample, or by a flow capillary.

27: The method according to claim 20, characterized in that the determination and characterization of particles is effected in a homogenous assay method without washing steps.

28: The method according to claim 20, characterized in that antibodies are used as fluorescence-labeled probes.

29: The method according to claim 20, characterized in that simultaneous analysis of the fluorescence-labeled probes in the sample is effected by emitting radiation of different wavelength or polarization plates.

30: The method according to claim 20, characterized in that the particles are pathological proteins, in particular prion proteins.

31: Use of the method according to claim 20 for pathogenic strain typing or for examining the relative binding of proteins from different species, in particular pathological proteins.

32: Use of the method according to claim 20 for the examination of degenerative diseases, in particular neurodegenerative diseases.

33: Use of the method according to claim 20 for the determination of particles selected from the group consisting of prion protein, APP, Tau, synuclein, protein having a polygluatamine sequence, such as huntingtin or fragments or derivatives thereof.

34: Use of the method according to claim 20 for the determination of subcellular particles, in particular for the phenotypical analysis of viral particles or for analysis of nucleic acids.
